(19) United States
(12) Patent Application Publication
(10) Pub. No.: US 2026/0107969 A1
(43) Pub. Date: Apr. 23, 2026

(54) AEROSOL GENERATING MATERIAL COMPRISING A SUBSTITUTED 3-(1-METHYLPYRROLIDIN-2-YL)PYRIDINE COMPOUND

(71) Applicant: RAI STRATEGIC HOLDINGS, INC., Winston-Salem, NC (US)

(72) Inventors: Kyle Ford, Pfafftown, NC (US); Brandon S. Darrow, Winston-Salem, NC (US); Michael R. Galloway, Winston-Salem, NC (US); Gary M. Dull, Lewisville, NC (US)

(21) Appl. No.: 19/361,772

(22) Filed: Oct. 17, 2025

Related U.S. Application Data
(60) Provisional application No. 63/708,976, filed on Oct. 18, 2024.
(60) Provisional application No. 63/880,572, filed on Sep. 12, 2025.

(51) Int. Cl. A24B 15/16; A24B 15/30; A24B 15/32; A24B 15/38; A24C 5/01; A24C 5/18; A24D 1/18; A24D 1/20; A24F 40/20; A61K 31/00; A61K 31/4045; A61K 31/465; A61K 31/522; A61K 36/48

(52) U.S. Cl. CPC A24B 15/16; A24B 15/301; A24B 15/303; A24B 15/32; A24B 15/385; A24C 5/01; A24C 5/1892; A24D 1/18; A24D 1/20; A24F 40/20; A61K 31/4045; A61K 31/465; A61K 31/522; A61K 31/658; A61K 36/48

(57) ABSTRACT

An aerosol generating material for use in an aerosol delivery device is provided, the aerosol generating material including a fibrous plant material, an aerosol former, and an active agent including at least a substituted 3-(1-methylpyrrolidin-2-yl)pyridine, a 3-(azetidin-2-yl)pyridine, or a 3-(azetidin-2-ylmethoxy)pyridine. The active agent is absorbed or adsorbed in or on the fibrous plant material. The disclosure further provides devices and aerosol provision systems incorporating such aerosol generating material.

### CROSS-REFERENCE TO RELATED APPLICATIONS

This application claims priority to U.S. Provisional Application No. 63/708,976, filed on Oct. 18, 2024, and claims priority to U.S. Provisional Application No. 63/880,572, filed on Sep. 12, 2025, each of which is incorporated herein by reference in its entirety and for all purposes.

### FIELD OF THE DISCLOSURE

The present disclosure relates to an aerosol generating material for use in aerosol provision systems such as smoking articles designed to deliver at least one substance to a user.

### BACKGROUND

Aerosol provision systems, and in particular, non-combustible aerosol provision systems have been proposed through the years as improvements upon, or alternatives to, smoking products that require combusting tobacco for use. These systems are generally designed to deliver at least one substance to a user, such as to satisfy a particular “consumer moment.” To this end, the substance may include constituents that impart a physiological effect on the user, a sensorial effect on the user, or both. The substance may be generally present in an aerosol-generating material that may contain one or more constituents of a range of constituents, such as active substances, flavors, aerosol formers, and other functional materials like fillers.

Aerosol provision systems include, for example, vapor products commonly known as “electronic cigarettes,” “e-cigarettes” or electronic nicotine delivery systems (ENDS), as well as heat-not-burn products including tobacco heating products (THPs) and carbon-tipped tobacco heating products (CTHPs). Many of these products take the form of a system including a device and a consumable, and it is the consumable that includes the material from which the substance to be delivered originates. Typically, the device is reusable, and the consumable is single use (although some consumables are refillable). Therefore, in many cases, the consumable is sold separately from the device, and often in a multipack. Moreover, subsystems and some individual components of devices or consumables may be sourced from specialist manufacturers. Alternatively, aerosol provision systems may be disposable, such that once the vapor forming material is depleted, the entire device is discarded.

Aerosol generating materials in such aerosol provision systems are typically heated, for example by a non-combustible aerosol provision system, to form an aerosol, which may be inhaled by a consumer. Aerosol generating materials may be made from various source materials, including tobacco material and/or non-tobacco materials.

### SUMMARY

The present disclosure is generally directed to aerosol generating materials for use in an aerosol delivery device. The aerosol generating materials generally comprise a fibrous plant material, an aerosol former, and an active agent absorbed or adsorbed in or on the fibrous plant material, the active agent comprising at least a substituted 3-(1-methylpyrrolidin-2-yl)pyridine, a 3-(azetidin-2-yl)pyridine, or a 3-(azetidin-2-ylmethoxy)pyridine.

The present disclosure includes, without limitation, the following embodiments.

Embodiment 1: An aerosol generating material comprising a fibrous plant material; an aerosol former; and an active agent absorbed or adsorbed in or on the fibrous plant material, the active agent comprising at least a compound having a structure according to Formula I, Formula II, or Formula III:

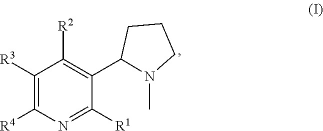

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

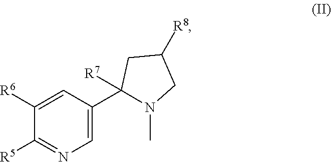

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen; or

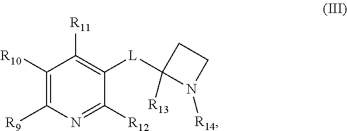

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

Embodiment 2: The aerosol generating material of Embodiment 1, wherein the active agent comprises a compound having a structure according to Formula I, and wherein R¹, R², and R³ are each H.

Embodiment 3: The aerosol generating material of Embodiment 1 or 2, wherein the active agent comprises a compound having a structure according to Formula I, and wherein R⁴ is optionally substituted C₁-C₆ alkyl, F, Cl, Br, OCH₃, OEt, or CN.

Embodiment 4: The aerosol generating material of Embodiment 3, wherein R⁴ is C₁-C₃ alkyl, such as wherein R⁴ is CH₃.

Embodiment 5: The aerosol generating material of Embodiment 1, wherein the active agent comprises a compound having a structure according to Formula II, wherein R⁵ and R⁶ are H; R⁷ is CH₃; and R⁸ is H.

Embodiment 6: The aerosol generating material of Embodiment 1, wherein the active agent comprises a compound having a structure according to Formula I, wherein R⁵, R⁶ and R⁷ are H, and R⁸ is CH₃.

Embodiment 7: The aerosol generating material of any one of Embodiments 1-6, comprising the fibrous plant material in an amount from about 70% to about 95% by weight, based on a total weight of the aerosol generating material on a dry weight basis.

Embodiment 8: The aerosol generating material any one of Embodiments 1-7, wherein the fibrous plant material comprises a tobacco material.

Embodiment 9: The aerosol generating material of any one of Embodiments 1-7, wherein the fibrous plant material comprises a non-tobacco botanical material.

Embodiment 10: The aerosol generating material of Embodiment 9, wherein the non-tobacco botanical material is a reconstituted non-tobacco botanical material.

Embodiment 11: The aerosol generating material of any one of Embodiments 1-7, wherein the fibrous plant material comprises a non-tobacco botanical material and the aerosol generating material further comprises a filler.

Embodiment 12: The aerosol generating material of Embodiment 11, wherein the filler comprises a tobacco material, wood fibers, wood pulp, or a combination thereof.

Embodiment 13: The aerosol generating material of any one of Embodiments 9 to 12, wherein the non-tobacco botanical material is selected from the group consisting of fennel, star anise, mint, Aspalathus linearis (rooibos), and combinations thereof.

Embodiment 14: The aerosol generating material of any one of Embodiments 1-13, wherein the aerosol generating material is substantially free of tobacco material.

Embodiment 15: The aerosol generating material of any one of Embodiments 1-14, wherein the aerosol generating material is substantially free of tea leaves, cassia seeds, and lotus leaves.

Embodiment 16: The aerosol generating material of any one of Embodiments 1-15, wherein the aerosol generating material is substantially free of 2-methyl-5-(1-methylpyrrolidin-2-yl)pyridine.

Embodiment 17: The aerosol generating material of any one of Embodiments 1-15, wherein the active agent further comprises a nicotine component, a cannabinoid, a terpene, caffeine, an amino acid, a vitamin, melatonin, a botanical extract, or a combination thereof.

Embodiment 18: The aerosol generating material of any one of Embodiments 1-15, wherein the active agent further comprises a nicotine component.

Embodiment 19: The aerosol generating material of any one of Embodiments 1-15, wherein the aerosol generating material is substantially free of a nicotine component.

Embodiment 20: The aerosol generating material of any one of Embodiments 1-19, further comprising a flavorant.

Embodiment 21: The aerosol generating material of any one of Embodiments 1-20, further comprising an organic acid.

Embodiment 22: The aerosol generating material of Embodiment 21, wherein the organic acid comprises lactic acid, levulinic acid, benzoic acid, citric acid, 2-methylbutyric acid, 2-methylvaleric acid, or a combination thereof.

Embodiment 23: The aerosol generating material of any one of Embodiments 1-22, wherein the aerosol former comprises water, polyhydric alcohols, polysorbates, sorbitan esters, fatty acids, fatty acid esters, waxes, cannabinoids, terpenes, sugar alcohols, or a combination thereof.

Embodiment 24: The aerosol generating material of any one of Embodiments 1-23, comprising the aerosol former in an amount from about 10% to about 20% by weight of the aerosol generating material.

Embodiment 25: The aerosol generating material of any one of Embodiments 1-24, wherein a ratio by weight of the aerosol former to the fibrous plant material is in a range from about 1:7 to about 1:3.

Embodiment 26: An aerosol generating rod comprising the aerosol generating material of any one of Embodiments 1-25.

Embodiment 27: An article comprising the aerosol generating material of any one of Embodiments 1 to 25 or the aerosol generating rod according to Embodiment 26.

Embodiment 28: The article of Embodiment 27, comprising an aerosol generating section comprising the aerosol generating material or the aerosol generating rod.

Embodiment 29: The article of Embodiment 27, comprising a wrapper circumscribing the aerosol generating material or the aerosol generating rod.

Embodiment 30: A delivery system comprising the aerosol generating material of any one of Embodiments 1 to 25, or the aerosol generating rod of Embodiment 26.

Embodiment 31: The delivery system of Embodiment 30, wherein the aerosol generating material is in the form of cut strips.

Embodiment 32: A method of preparing an aerosol generating material comprising a fibrous plant material; an aerosol former; optionally, a binder, a filler, or both a binder and a filler; and an active agent absorbed or adsorbed in or on the fibrous plant material, the active agent comprising at least a substituted 3-(1-methylpyrrolidin-2-yl)pyridine, a 3-(azetidin-2-yl)pyridine, or a 3-(azetidin-2-ylmethoxy)pyridine having a structure according to Formula I, Formula II, or Formula III, the method comprising: providing a sheet of aerosol generating material, the sheet comprising a fibrous plant material; an aerosol former; optionally, a binder, a filler, or both a binder and a filler; and applying the substituted 3-(1-methylpyrrolidin-2-yl)pyridine to the sheet of aerosol generating material.

Embodiment 33: A method of preparing a plurality of aerosol generating rods, comprising: a) providing an aerosol generating material comprising a fibrous plant material and an aerosol former, the aerosol generating material being in the form of a sheet, an extrudate, or a shredded material (such as any of the aerosol generating materials of Embodiments 1 to 25); b) conveying the aerosol generating material to a rod formation device; c) applying a substituted 3-(1-methylpyrrolidin-2-yl)pyridine, a 3-(azetidin-2-yl)pyridine, or a 3-(azetidin-2-ylmethoxy)pyridine to the aerosol generating material as the aerosol generating material is conveyed to the rod formation device; d) forming the aerosol generating material into an enwrapped rod having an outer wrapper circumferentially wrapped around the aerosol generating material; and e) cutting the enwrapped rod into discrete lengths to form the plurality of aerosol generating rods.

Embodiment 34: The method of Embodiment 33, further comprising attaching a filter element to each of the plurality of aerosol generating rods.

Embodiment 35: The method of Embodiment 33, wherein the substituted 3-(1-methylpyrrolidin-2-yl)pyridine, a 3-(azetidin-2-yl)pyridine, or a 3-(azetidin-2-ylmethoxy)pyridine has a structure according to Formula I, Formula II, or Formula III:

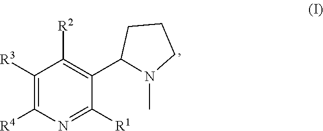

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

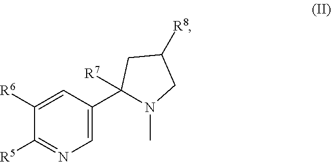

wherein:
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen; or

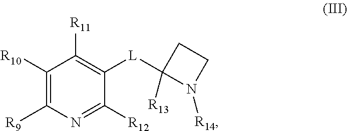

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

Embodiment 36: The method of any one of Embodiments 33 to 35, wherein the applying comprises metered dosing of the substituted 3-(1-methylpyrrolidin-2-yl)pyridine, a 3-(azetidin-2-yl)pyridine, or a 3-(azetidin-2-ylmethoxy)pyridine in a range from about 0.1 to about 5 mg per aerosol generating rod, such as a range of from about 0.3 to about 2 mg or about 0.5 mg to about 1.5 mg per aerosol generating rod, and optionally wherein the variance in metered dosing for each aerosol generating rod is no more than +/−10%.

Embodiment 37: The aerosol generating material of Embodiment 1, wherein the active agent comprises at least a compound having a structure according to Formula II; optionally, wherein R⁵ is optionally substituted C₁-C₆ alkyl, F, Cl, Br, OCH₃, OCH₂CH₃, or CN; and R⁶ is H; or optionally, wherein R⁵ is H; and R⁶ is optionally substituted C₁-C₆ alkyl, F, Cl, Br, OCH₃, OCH₂CH₃, or CN.

Embodiment 38: The aerosol generating material of Embodiment 1, wherein the active agent comprises at least a compound having a structure according to Formula III; optionally, wherein R⁹ is H or CH₃; and R¹⁰, R¹¹, and R¹² are each H.

These and other features, aspects, and advantages of the present disclosure will be apparent from a reading of the following detailed description together with the accompanying Figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

### BRIEF DESCRIPTION OF THE DRAWINGS

Having thus described aspects of the disclosure in the foregoing general terms, reference will now be made to the accompanying figures, which are not necessarily drawn to scale, and wherein:

FIG. 1 is a side-on cross-sectional view of an article comprising the aerosol generating material according to a non-limiting embodiment of the disclosure.

FIG. 2 is a perspective illustration of a non-combustible aerosol provision device for generating aerosol from an article according to a non-limiting embodiment of the disclosure.

FIG. 3 is a schematic representation of an example manufacturing process for a consumable according to a non-limiting embodiment of the disclosure.

FIG. 4 is a schematic representation of an example consumable assembly process according to a non-limiting embodiment of the disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements.

As used in this specification and the claims, the singular forms “a,” “an,” and “the” include plural referents unless the context clearly dictates otherwise.

Reference to “dry weight percent” or “dry weight basis” refers to weight on the basis of dry ingredients (i.e., all ingredients except water). All weight percent values herein are dry weight percent unless otherwise indicated.

The terms ‘upstream’ and ‘downstream’ used herein are relative terms defined in relation to the direction of mainstream aerosol drawn though an article or device in use.

Unless otherwise defined herein, by “substantially free” it is meant that the noted component (e.g., nicotine or a specific botanical material) has not been intentionally added, beyond trace amounts that may be present e.g., as an impurity in another component, or small amounts which may be present in certain flavor packages. For example, some embodiments can have less than 0.01% by weight of the noted component, or less than 0.001%, or even 0% by weight of the noted component, based on the total weight of the aerosol generating material. In some embodiments, the aerosol generating material is completely free of the noted component (i.e., having 0% or as having an amount below the limit of detection).

The present disclosure is generally directed to aerosol generating materials comprising a fibrous plant material, an aerosol former, and an active agent absorbed or adsorbed in or on the fibrous plant material, and to various aerosol delivery devices comprising such aerosol generating materials. The aerosol generating materials are capable of generating an aerosol, for example a suspension of liquid droplets or particulates in a gas, when heated, irradiated, or energized in any suitable manner. Accordingly, the disclosure provides generating materials adapted for use in an aerosol delivery device. Such aerosol generating materials and aerosol delivery devices comprising them are described further herein below.

### Fibrous Plant Material

An aerosol generating material as disclosed herein comprises a fibrous plant material. In some embodiments, the fibrous plant material comprises a botanical material. As used herein, the term “botanical” includes any material derived from plants including, but not limited to, extracts, leaves, bark, fibers, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens.

A fibrous plant material comprising a botanical material may supplement the flavor profile of any aerosol produced. For example, a botanical material may be selected which has a relatively neutral flavor profile. A neutral flavor profile may be described as a flavor profile which contains few intense flavors and/or one which readily accepts the loading of top flavors, without significantly affecting their perception. It may be advantageous to be able to produce an aerosol which has a similarly neutral flavor profile to that of reconstituted tobacco, but without using tobacco material.

In some embodiments, the botanical material may be any non-tobacco botanical material. For example, the material may be derived from species which are members of the Asteracae family, the Fabaceae family, the Myrtaceae family, Apiaceae family, Camellia taliensis, the Solanaceae family, the Brassicaceae family, the Caricaceae family, the Asclepiadaceae family, the Equisetaceae family, the Oleaceae family, the Lamiaceae family, and tisanes. For example, the non-tobacco botanical material may be selected from the Matricaria species, such as chamomile; the Pimpinella anisum species, such as anise; the Foeniculum vulgare species, such as fennel; jasmine; lavender; cloves; eucalyptus, and the species Aspalathus linearis, such as rooibos (red or green).

In some embodiments, the non-tobacco botanical material is selected from a botanical material which comprises favorable aroma properties for use in a non-combustible aerosol provision system. For example, the non-tobacco botanical material may comprise relatively few aroma compounds compared to traditional tobacco material; therefore, an aerosol produced from a non-tobacco botanical material may have a different profile of volatile compounds compared to an aerosol produced from a tobacco material. The non-tobacco botanical material may deliver an aerosol which is considered favorable by a consumer of tobacco-based delivery systems. In some embodiments, a non-tobacco botanical material may produce an aerosol, when heated, with a sensorial experience that is comparable to that provided by a conventional combustible product, such as a cigarette. In some embodiments, the non-tobacco botanical material is selected from seed-producing plants which do not develop persistent woody tissue and which are often valued for their medicinal or sensorial characteristics. In some embodiments, it may be preferable to provide an aerosol generating material for use in a non-combustible aerosol provision system which does not comprise any tobacco plant material.

In some embodiments, the non-tobacco botanical material is selected from the group consisting of eucalyptus, star anise, hemp, cocoa, cannabis, fennel, lemongrass, peppermint, spearmint, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrate c.v., Mentha piperita c.v, Mentha spicata crispa, Mentha cardifolia, Mentha longifolia, Mentha suaveolens variegata, Mentha pulegium, Mentha spicata c.v., and Mentha suaveolens.

In some embodiments, the fibrous plant material comprises rooibos.

In some embodiments, the fibrous plant material is selected from the group consisting of fennel, star anise, mint, Aspalathus linearis (rooibos), and combinations thereof.

In some embodiments, the aerosol generating material does not comprise rooibos or is substantially free of rooibos. In some embodiments, the aerosol generating material comprises a botanical material which is not rooibos. For example, the aerosol generating material may comprise any botanical material disclosed herein, apart from rooibos, for example the botanical material may be selected from star anise, mint, and fennel, and mixtures thereof.

In some embodiments, the aerosol generating material is substantially free of tobacco material. As used herein, the term “tobacco material” refers to any material comprising tobacco or derivatives or substitutes thereof, including tobacco, tobacco derivatives, expanded tobacco, reconstituted tobacco or tobacco substitutes. It is understood that a nicotine extract, derived from tobacco material, is not considered to be a “tobacco material.”

In some embodiments, the aerosol generating material is substantially free of tea leaves, cassia seeds, and lotus leaves.

In some embodiments, the non-tobacco botanical material is a reconstituted non-tobacco botanical material. Reference to a “reconstituted” material refers to a plant material that has been treated with a solvent to form a slurry or extract, with example reconstitution processes including papermaking processes and band cast processes. The reconstitution process typically includes the addition of one or more additives to the slurry or extract, such as binding agents, flavorants, and the like, followed by formation of a sheet material that is subsequently dried. In some processes, an extract is removed from the plant material and separately treated. The extract or a portion thereof can be recombined with the remaining plant pulp material as part of the reconstitution process. Example reconstitution processes, which can be applied to tobacco or non-tobacco plant materials, are described in U.S. Pat. No. 6,705,325 to Hicks et al.; U.S. Pat. No. 6,827,087 to Wanna et al.; and U.S. Patent Application Publication Nos. 2004/0177856 to Monsalud, Jr. et al. and 2005/0056294 to Wanna et al., which are all incorporated herein by reference.

The amount of fibrous plant material present in the aerosol generating material may vary. In some embodiments, the fibrous plant material is present in an amount from about 10% to about 95% by weight, based on a total weight of the aerosol generating material on a dry weight basis.

In some embodiments, the fibrous plant material comprises first and second fibrous plant materials. In some embodiments, the first and second fibrous plant materials are different. When the first and second fibrous plant materials are different, the properties of the aerosol generating material, e.g., the aerosol generated, may be modified. For example, two different fibrous plant materials could be combined which complement each other and provide an aerosol that is particularly pleasant/favorable to a consumer.

In some embodiments, the first fibrous plant material comprises a botanical material as described herein and the second fibrous plant material comprises wood fibers/wood pulp. Used herein, wood fiber and wood pulp may be used to describe a cellulose material derived from a cellulose material which has little, or substantially no, noticeable aroma. For example, wood fiber and wood pulp may be of similar nature to wood fiber/wood pulp used to make paper. Wood fiber and wood pulp are obtained from a non-tobacco material.

In some embodiments, a ratio between the first fibrous plant material and the second fibrous plant material may be from about 10:1 to about 2:1. For example, the ratio may be from about 9:1 to about 3:1; such as from about 8:1 to about 4:1; such as from about 7:1 to about 4:1; such as from about 6:1 to about 4:1. In some embodiments, a ratio between the first fibrous plant material and the second fibrous plant material may be about 5:1.

In some embodiments, modifying the amount of a first plant fibrous material, comprising, for example, a botanical material; and a second fibrous plant material, comprising, for example, wood fibers/wood pulp, enables the aerosol to be modified/tailored to a specific desirable profile. For example, one may increase the relative amount of a botanical material, compared to wood fiber/wood pulp, therefore increasing the amount of aroma compounds derived from the botanical material in an aerosol.

### Filler

In some embodiments, the aerosol generating material as described herein comprises a filler component. The filler component is generally a non-tobacco component; that is, a component that does not include ingredients originating from tobacco. The filler component may be a non-tobacco fiber such as wood fiber or pulp or wheat fiber. The filler component may also be an inorganic material such as chalk, perlite, vermiculite, diatomaceous earth, colloidal silica, magnesium oxide, magnesium sulphate, magnesium carbonate. The filler component may also be a non-tobacco cast material. In some embodiments, the filler comprises a tobacco material. In some embodiments, the filler comprises wood fibers, wood pulp, or a combination thereof. In some embodiments, the filler comprises a tobacco material, wood fibers, wood pulp, or a combination thereof. In some embodiments, the filler does not include a tobacco material.

The filler component may be present in an amount from about 0 to about 20% by weight, or in an amount of from about 1 to about 10% by weight of the aerosol generating material. In some embodiments, the aerosol generating material comprises the filler component in an amount between about 5% and about 10% by weight of the aerosol generating material. In some embodiments, the filler component is absent.

### Binder

In some embodiments, the aerosol generating material as described herein comprises a binder. When present, the binder generally comprises an alginate, pectin, starch (and derivatives), cellulose (and derivatives), gum, silica or silicone compounds, clay, polyvinyl alcohol, or combinations thereof. For example, in some embodiments, the binder comprises one or more of alginates, pectins, hydroxyethyl cellulose, hydroxypropyl cellulose, carboxymethylcellulose (CMC), pullulan, xanthan gum, guar gum, carrageenan, agarose, acacia gum, fumed silica, PDMS, sodium silicate, kaolin and polyvinyl alcohol. In some embodiments, the binder comprises alginate and/or pectin or carrageenan. In some embodiments, the binder comprises CMC.

### Aerosol Former

An aerosol generating material as disclosed herein comprises an aerosol former. Generally, the aerosol former includes one or more constituents capable of forming an aerosol, for example, by thermal or mechanical vaporization. An aerosol former may promote the generation of an aerosol by promoting an initial vaporization and/or the condensation of a gas to an inhalable solid and/or liquid aerosol. In some embodiments, an aerosol former may improve the delivery of flavor from the aerosol generating material. The aerosol former has been found to improve the sensory performance of an article for use with an aerosol generation device comprising the aerosol generating material, by helping to transfer compounds such as flavor compounds from the fibrous material to the consumer. In some embodiments, the aerosol former is flavored and/or comprises a flavor as described herein.

In general, any suitable aerosol former may be included in the aerosol generating material. Non-limiting examples of suitable classes of aerosol formers include water, polyhydric alcohols, polysorbates, sorbitan esters, fatty acids, fatty acid esters, triacetin, waxes, sugar alcohols, and combinations thereof. The particular choice of aerosol former(s) may depend on factors such as the method of aerosol formation, the appearance and volume of the aerosol, the desired density of the aerosol, and the like. In some embodiments, the aerosol former may include, but is not limited to, polyols and non-polyols such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate.

In some embodiments, the aerosol former includes one or more polyhydric alcohols. Examples of polyhydric alcohols include glycerol, propylene glycol, and other glycols such as 1,3-propanediol, diethylene glycol, triethylene glycol, and polyethylene glycols (e.g., PEG molecules with weight average molecular weight range of about 200 to about 2,000 Da). In some embodiments, the aerosol former comprises one or more polyhydric alcohols. In some embodiments, the one or more polyhydric alcohols are selected from the group consisting of glycerol, propylene glycol, 1,3-propanediol, diethylene glycol, triethylene glycol, a polyethylene glycol, and combinations thereof. In some embodiments, the polyhydric alcohol is selected from the group consisting of glycerol, propylene glycol, and mixtures thereof. In some embodiments, the aerosol generating material is substantially free of propylene glycol. In some embodiments, the aerosol generating material is substantially free of glycerin.

In some embodiments, the aerosol former comprises one or more polysorbates, one or more sorbitan esters, one or more fatty acids, one or more fatty acid esters, triacetin, one or more waxes, one or more sugar alcohols, or combinations thereof. Examples of polysorbates include, but are not limited to, Polysorbate 60 (polyoxyethylene (20) sorbitan monostearate, Tween® 60) and Polysorbate 80 (polyoxyethylene (20) sorbitan monooleate; Tween® 80). Examples of sorbitan esters include, but are not limited to, sorbitan monolaurate, sorbitan monostearate (Span® 60), sorbitan monooleate (Span® 20), and sorbitan tristearate (Span® 65). Examples of fatty acids include, but are not limited to, butyric, propionic, valeric, oleic, linoleic, stearic, myristic, and palmitic acids. Examples of fatty acid esters include, but are not limited to, alkyl esters, monoglycerides, diglycerides, and triglycerides. Examples of monoglycerides include monolaurin and glycerol monostearate. Examples of triglycerides include triolein, tripalmitin, tristearate, glycerol tributyrate, and glycerol trihexanoate. Examples of waxes include, but are not limited to, carnauba, beeswax, candelilla. Examples of sugar alcohols include, but are not limited to, sorbitol, erythritol, mannitol, maltitol, isomalt, and xylitol. In some embodiments, the aerosol former further comprises erythritol, meso-erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, or combinations thereof.

In some embodiments, the aerosol former comprises water, polyhydric alcohols, polysorbates, sorbitan esters, fatty acids, fatty acid esters, waxes, cannabinoids, terpenes, sugar alcohols, or a combination thereof.

In some embodiments, the aerosol former is selected from the group consisting of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, and mixtures thereof.

In some embodiments, the aerosol former comprises a combination of propylene glycol and glycerol. The relative amounts of propylene glycol and glycerol may vary and may be expressed as a weight ratio. In some embodiments, the aerosol former comprises propylene glycol and glycerol in a ratio by weight from about 1:3 to about 3:1, such as about 1:3, about 1:2, about 1:1, about 2:1, or about 3:1.

The amount of aerosol former present in the aerosol generating material may vary. The amount of aerosol former present may be expressed as a ratio by weight of aerosol former to fibrous plant material. In some embodiments, a ratio of aerosol former to fibrous material is from about 1:7 to about 1:3. For example, from about 1:6 to about 1:4, such as about 1:5. When the ratio of aerosol former to fibrous plant material is within this range, the aerosol generating material exhibits favorable storage properties and generates a favorable aerosol.

### Active Agent

The aerosol generating material as described herein comprises an active agent absorbed or adsorbed in or on the fibrous plant material. The active agent comprises at least a compound having a structure according to Formula I, Formula II, or Formula III:

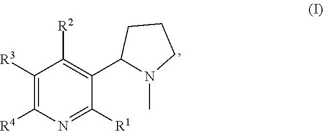

wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or

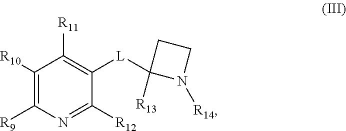

wherein.
   R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
   R⁷ is selected from the group consisting of hydrogen and CH₃;
   R⁸ is selected from the group consisting of hydrogen and C₁-C₃ alkyl; and
   at least one of R⁷ and R⁸ is not hydrogen; or

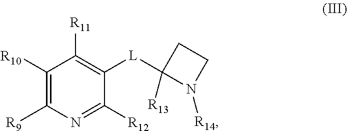

wherein:
   L is a bond or —OCH₂—*, where the asterisk indicates an attachment point to the azetidine ring;
   R⁹, R¹⁰, R¹¹, and R¹² are each independently selected from the group consisting of hydrogen, alkyl, alkoxy, halogen, and cyano;
   R¹³ is H or CH₃; and
   R¹⁴ is H or CH₃.

In some embodiments, the substituted 3-(1-methylpyrrolidin-2-yl)pyridine has a structure according to Formula I. Substituted 3-(1-methylpyrrolidin-2-yl)pyridines with various R¹, R², R³, and R⁴ substituents have been reported previously. See for example, U.S. Pat. Nos. 4,321,387, 4,155,909; 5,015,741, 5,138,062, and 5,703,100, each of which is incorporated by reference herein and describe such substituted 3-(1-methylpyrrolidin-2-yl)pyridines, their synthesis, and pharmacological properties. Substituted 3-(1-methylpyrrolidin-2-yl)pyridines and their pharmacological profiles have also been disclosed in Wang et al., Drug Development Research 1998, Volume 45, Issue 1, Pages 10-16; and Dukat et al. European Journal of Medicinal Chemistry 1999, 34(1): 31-40.

In some embodiments, the substituted 3-(1-methylpyrrolidin-2-yl)pyridine has a structure according to Formula I, wherein R¹, R², and R³ are each H.

In some embodiments, R⁴ is optionally substituted C₁-C₆ alkyl, F, Cl, Br, OCH₃, OEt, or CN. In some embodiments, R⁴ is C₁-C₃ alkyl, such as CH₃.

In some embodiments, R¹, R², and R³ are each H, and R⁴ is Me. In such embodiments, the compound of Formula I may be referred to as 2-methyl-5-(1-methylpyrrolidin-2-yl)pyridine.

The pharmacology of 2-methyl-5-(1-methylpyrrolidin-2-yl)pyridine has been reported in, for example, Dukat et al. European Journal of Medicinal Chemistry, Volume 31, Issue 11, 1996, Pages 875-888 (incorporated herein by reference), and the pharmacological profile of the (S)-enantiomer of 2-methyl-5-(1-methylpyrrolidin-2-yl)pyridine in the form of the benzoate salt (CAS 2861225-70-7; referred to as Imotine™) is discussed in Carmines et al, Poster #6; 76^{th} TSRC Conference 2023, Norfolk, VA, USA).

In some embodiments, the aerosol generating material is substantially free of 2-methyl-5-(1-methylpyrrolidin-2-yl)pyridine.

In some embodiments, the substituted 3-(1-methylpyrrolidin-2-yl)pyridine has a structure according to Formula II. Certain substituted 3-(1-methylpyrrolidin-2-yl)pyridines with various R⁵, R⁶, R⁷, and R⁸ substituents have been reported previously. See for example, U.S. Pat. Nos. 4,321,387, 4,155,909; 5,015,741, 5,138,062, and 5,703,100, each of which is incorporated by reference herein and describe example substituted 3-(1-methylpyrrolidin-2-yl)pyridines, their synthesis, and pharmacological properties. Certain substituted 3-(1-methylpyrrolidin-2-yl)pyridines and their pharmacological profiles have also been disclosed in Wang et al., Drug Development Research 1998, Volume 45, Issue 1, Pages 10-16; Dukat et al. European Journal of Medicinal Chemistry 1999, 34(1): 31-40; Lin et al., J. Med. Chem. (1994), 37, 3542-3553.

In some embodiments, R⁵ and R⁶ are H; R⁷ is CH₃; and R⁸ is H. In such embodiments, the compound of Formula II may be referred to as 3-(1,2-dimethylpyrrolidin-2-yl)pyridine, or 2′-methyl-5-(1-methylpyrrolidin-2-yl)pyridine, and has a structure:

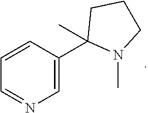

The compound 3-(1,2-dimethylpyrrolidin-2-yl)pyridine is known in the literature, and has a Chemical Abstracts Registry Number of 220650-38-4. The synthesis of this compound has been reported in Rouchaud et al., J Het Chem 2012, 49(1), 161-166; Wang et al., Drug Dev Res 1998, 45(1), 10-16; Secor et al., Tetrahedron Lett. (1981), 22(33), 3151-3154; US Patent Application Publication No. 2013/0157995; PCT Application Publication No. WO2012/031220; and U.S. Pat. No. 9,440,948, each of which are incorporated by reference herein with respect to the synthesis of 3-(1,2-dimethylpyrrolidin-2-yl)pyridine.

In some embodiments, R⁵ and R⁶ are H; R⁷ is H; and R⁸ is CH₃. In such embodiments, the compound of Formula II may be referred to as 3-(1,4-dimethylpyrrolidin-2-yl)pyridine, or 4′-methyl-5-(1-methylpyrrolidin-2-yl)pyridine, and has a structure:

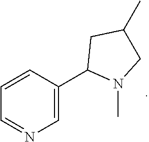

The compound 3-(1,4-dimethylpyrrolidin-2-yl)pyridine is known in the literature, has a Chemical Abstracts Registry Number of 74805-00-8, and is commercially available from, for example, Enamine Stock Building Blocks and Aurora Building Blocks. The synthesis of this compound has been reported in U.S. Pat. No. 9,440,948; and EP Patent No. 559495, each of which are incorporated by reference herein with respect to the synthesis of 3-(1,4-dimethylpyrrolidin-2-yl)pyridine.

In some embodiments, R⁵ is CH₃; R⁶ is H; R⁷ is CH₃; and R⁸ is H. In such embodiments, the compound of Formula II may be referred to as 5-(1,2-dimethylpyrrolidin-2-yl)-2-methylpyridine, and has a structure:

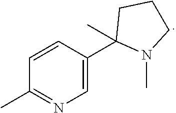

The compound 5-(1,2-dimethylpyrrolidin-2-yl)-2-methylpyridine may be readily synthesized according to known reactions. For example, commercially available 2-methyl-5-(2-methylpyrrolidin-2-yl)pyridine (Chemical Abstracts Registry Number of 1528955-30-7; Aurora Building Blocks, Adlab Chemicals Building Blocks) can be N-methylated with formaldehyde and formic acid, or alternatively with formaldehyde and a reducing agent such as sodium cyanoborohydride to afford 5-(1,2-dimethylpyrrolidin-2-yl)-2-methylpyridine. Alternatively, 5-(1,2-dimethylpyrrolidin-2-yl)-2-methylpyridine may be synthesized by lithiation of 2-methyl-5-bromopyridine, reaction of the lithiated pyridine with N-methylpyrrolidone, and addition of methyl lithium to the perchlorate salt of the resulting imine. This reaction sequence is shown below in Scheme 1.

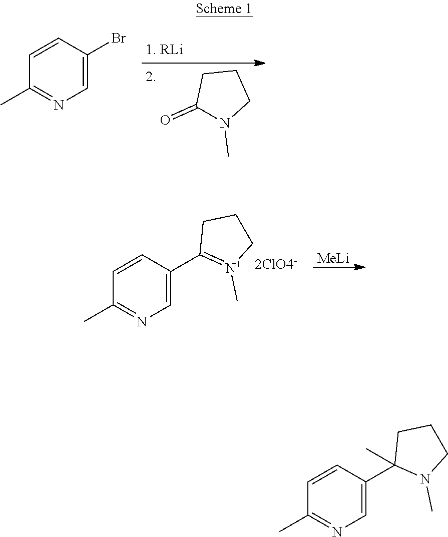

In some embodiments, R⁵ is H or CH₃, R⁶ is H, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from

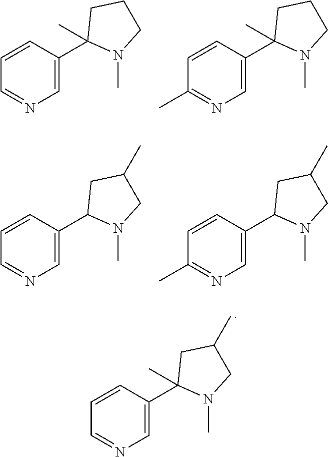

In some embodiments, R⁵ is H, R⁶ is F, CH₃, or OCH₃, R⁷ is H or CH₃, and R⁸ is H or CH₃, provided that at least one of R⁷ and R⁸ is CH₃. Accordingly, in some embodiments, the substituted 3-(1-methylpyrrolidin-2-yl)pyridine has a structure selected from:

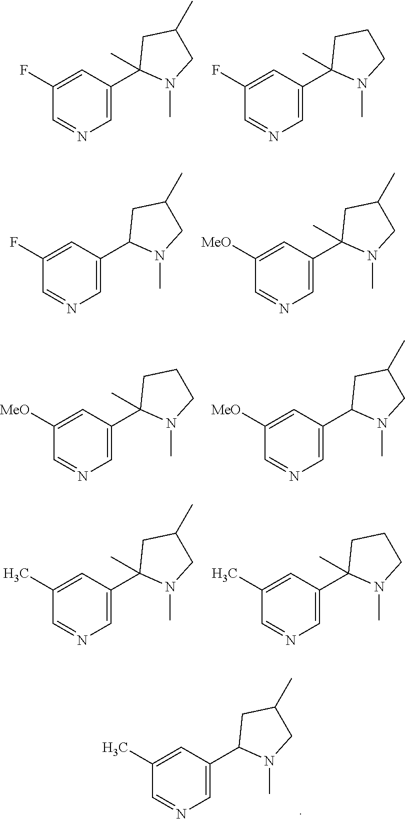

A substituted 3-(1-methylpyrrolidin-2-yl)pyridine and bearing one or more substituents on the pyrrolidine ring as described herein may be present as a single enantiomer or as a mixture of enantiomers. In some embodiments, the substituted 3-(1-methylpyrrolidin-2-yl)pyridine bearing one or more substituents on the pyrrolidine ring is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer. In some embodiments, the aerosol generating material predominantly comprises the substituted 3-(1-methylpyrrolidin-2-yl)pyridine bearing one or more substituents on the pyrrolidine ring in the (R)-configuration, for example, about 90% or more of the total quantity of substituted 3-(1-methylpyrrolidin-2-yl)pyridine bearing one or more substituents on the pyrrolidine ring present is in the (R)-configuration. In some embodiments, the aerosol generating material predominantly comprises the substituted 3-(1-methylpyrrolidin-2-yl)pyridine bearing one or more substituents on the pyrrolidine ring in the (S)-configuration, for example, about 90% or more of the total quantity of substituted 3-(1-methylpyrrolidin-2-yl)pyridine present is in the (S)-configuration. In some embodiments, the aerosol generating material comprises 95% or more of the (S)-configuration of the substituted 3-(1-methylpyrrolidin-2-yl)pyridine, based on the total amount of substituted 3-(1-methylpyrrolidin-2-yl)pyridine present.

In some embodiments, the substituted 3-(1-methylpyrrolidin-2-yl)pyridine bearing one or more substituents on the pyrrolidine ring is non-racemic, and has one of the following structures:

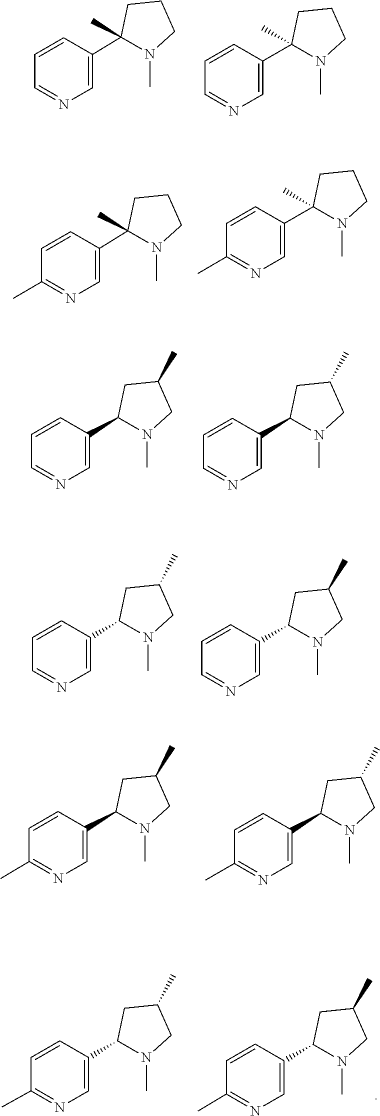

In some embodiments, the substituted 3-(1-methylpyrrolidin-2-yl)pyridine bearing one or more substituents on the pyrrolidine ring is non-racemic, and has a structure selected from:

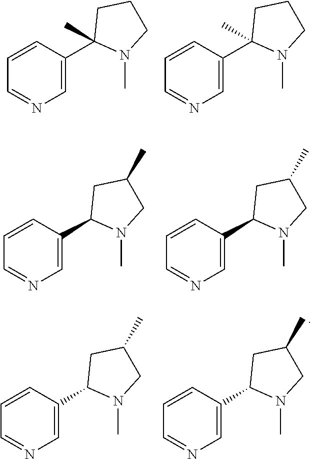

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of various substituted 3-(1-methylpyrrolidin-2-yl)pyridines such as those described herein has been reported in, for example, Lin et al., J. Med. Chem., 1994, 37, 3542-3553; Dukat et al. European Journal of Medicinal Chemistry, 31(11), 1996, 875-888; U.S. Pat. No. 9,440,948; Wang et al., Drug Dev Res 1998, 45(1), 10-16 (each of which is incorporated herein by reference), among many others. Generally, small substituents such as methyl groups are well tolerated at the 2′ or 4′ positions of the nicotine pyrrolidine ring, and small substituents such as alkyl, halogen, alkoxy, and the like are well tolerated at the 5 or 6 position of the nicotine pyridine ring. For example, 3-(1-methylpyrrolidin-2-yl)pyridines bearing a methyl substituent at the 2′ or 4′ position are equipotent or even more potent than nicotine with respect to binding affinity to the nicotinic acetylcholine receptor, and are expected to preserve the pharmacological effects of nicotine in vivo. See, for example U.S. Pat. No. 5,278,176, Lin et al., J. Med. Chem., 1994, 37, 3542-3553, and Wang et al., Drug Dev Res 1998, 45(1), 10-16.

Without wishing to be bound by any particular theory, it is believed that certain substitutions for hydrogen on the pyridine ring and/or pyrrolidine rings of the substituted 3-(1-methylpyrrolidin-2-yl)pyridine compound preserve the general pharmacological profile and physiological effects of nicotine while offering the potential for one or more of greater potency, longer half-life, reduced product consumption, more rapid and/or complete absorption, greater bioavailability, and the like. Particularly, it is believed that in some embodiments, substituted 3-(1-methylpyrrolidin-2-yl)pyridines of the disclosure are readily absorbed through membranes such as pulmonary alveoli, oral mucosa, and the like by virtue of their lipophilicity. Lipophilicity is conveniently measured in terms of log P, the partition coefficient of a molecule between a lipophilic phase and an aqueous phase, usually octanol and water, respectively. Accordingly, in some embodiments, the substituted 3-(1-methylpyrrolidin-2-yl)pyridine of Formula I or II has a calculated or experimental log P of about 1 or greater, where log P is the log₁₀ of the partitioning coefficient of the substituted 3-(1-methylpyrrolidin-2-yl)pyridine between octanol and water. Log P values may be measured experimentally according to protocols well known to one of skill in the art. Alternatively, log P values may be calculated using commercially available software. In some embodiments, the substituted 3-(1-methylpyrrolidin-2-yl)pyridine of Formula I or II has a calculated log P from 1 to about 2, such as 1.1, 1.2, 1.3, 1.4, 1.5, 1.6, 1.7, 1.8, or 1.9. In some embodiments, the substituted 3-(1-methylpyrrolidin-2-yl)pyridine of Formula I or II has a calculated log P from about 1.2 to about 1.7. In some embodiments, the substituted 3-(1-methylpyrrolidin-2-yl)pyridine having a calculated log P from about 1.2 to about 1.7 bears one or more lipophilic substituents on the pyridine ring, such as C₁-C₆ alkyl, or C₁-C₃ alkyl. In some embodiments, the lipophilic substituent is methyl, and is present at the 2, 4, 5, or 6 position of the pyridine ring. In some embodiments, the lipophilic substituted 3-(1-methylpyrrolidin-2-yl)pyridine is 2-methyl-5-(1-methylpyrrolidin-2-yl)pyridine.

The quantity of substituted 3-(1-methylpyrrolidin-2-yl)pyridine (e.g., 2-methyl-5-(1-methylpyrrolidin-2-yl)pyridine) present in the aerosol generating material may vary. Typically, the substituted 3-(1-methylpyrrolidin-2-yl)pyridine (e.g., 2-methyl-5-(1-methylpyrrolidin-2-yl)pyridine, calculated as the free base) is present in a concentration of at least about 0.001% by weight of the aerosol generating material, such as in a range from about 0.01% to about 10%. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 10% by weight, such as, e.g., from about from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the aerosol generating material. In some embodiments, the substituted 3-(1-methylpyrrolidin-2-yl)pyridine (e.g., 2-methyl-5-(1-methylpyrrolidin-2-yl)pyridine) is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the aerosol generating material. In some embodiments, the aerosol generating material comprises 2-methyl-5-(1-methylpyrrolidin-2-yl)pyridine in an amount from about 0.01 to about 1% by weight, such as from about 0.1 to about 0.75% by weight, based on the total weight of the aerosol generating material.

The substituted 3-(1-methylpyrrolidin-2-yl)pyridine (e.g., 2-methyl-5-(1-methylpyrrolidin-2-yl)pyridine) may be present in the aerosol generating material as the free base, as a salt with a suitable acid, in the form of an ion pair with an organic acid, or a combination thereof. Each of these forms is described further herein below.

### 3-(azetidin-2-yl)pyridines and 3-(azetidin-2-ylmethoxy)pyridines

In some embodiments, the active agent comprises an optionally substituted 3-(azetidin-2-yl)pyridine or an optionally substituted 3-(azetidin-2-ylmethoxy)pyridine. As used herein, the term “substituted 3-(azetidin-2-yl)pyridine” refers to a compound having a 3-(azetidin-2-yl)pyridine scaffold and bearing one or more non-hydrogen substituents on the azetidine ring, and optionally on the pyridine ring. As used herein, the term “substituted 3-(azetidin-2-ylmethoxy)pyridine” refers to a compound having a 3-(azetidin-2-ylmethoxy)pyridine scaffold and bearing one or more non-hydrogen substituents on the azetidine ring, and optionally on the pyridine ring.

In some embodiments, the 3-(azetidin-2-yl)pyridine or 3-(azetidin-2-ylmethoxy)pyridine has a structure according to Formula III:

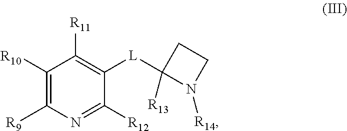

wherein:
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

In some embodiments, L is a bond.

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-yl) pyridine is 3-(azetidin-2-yl)pyridine, and has a structure:

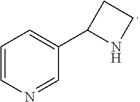

The compound 3-(azetidin-2-yl)pyridine is known in the literature. The synthesis of this compound has been reported in JOC 1979, 44(18), 3136; Med Chem Res (1993) 2:552-5633; in International Patent Application Publication No. WO2012/031220, and in U.S. Pat. Nos. 4,163,855 and 4,163,856, all of which are incorporated herein in their entireties.

In some embodiments, the 3-(azetidin-2-yl) pyridine is 3-(1-methylazetidin-2-yl)pyridine, having the structure:

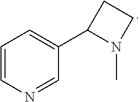

The compound 3-(1-methylazetidin-2-yl)pyridine is known in the literature. The synthesis of this compound has been reported in International Patent Application Publication No. WO2012/031220, previously incorporated by reference herein.

In some embodiments, the 3-(azetidin-2-yl) pyridine has a structure selected from the group consisting of:

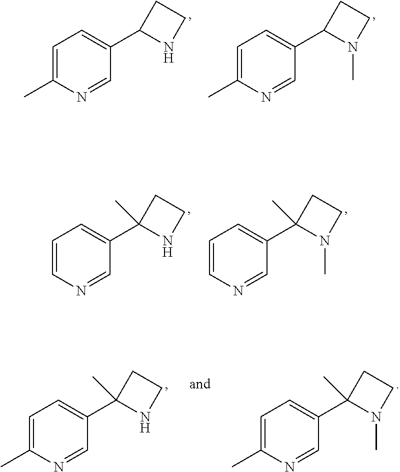

Such compounds are either known, or may be readily prepared according to adaptations of methods utilized for preparation of related 3-(azetidin-2-yl) pyridines and 3-(1-methylpyrrolidin-2-yl)pyridines described herein above. See, e.g., U.S. Pat. No. 4,163,855, previously incorporated by reference herein. The compound 5-(2-azetidinyl)-2-methylpyridine is known in the literature and has a Chemical Abstracts Registry (CAS) Number of 1270467-65-6, and the R- and S-enantiomers have CAS numbers 1213081-15-2 and 1212969-96-4, respectively.

In some embodiments, the aerosol generating material comprises a 3-(azetidin-2-ylmethoxy)pyridine (i.e., L is —OCH₂—*).

In some embodiments, R⁹ is CH₃, F, Cl, Br, OCH₃, OEt, or CN.

In some embodiments, R⁹ is H or CH₃; and R¹⁰, R¹¹, and R¹² are each H.

In some embodiments:
R¹³ and R¹⁴ are both H;
R¹³ and R¹⁴ are both CH₃;
R¹³ is H and R¹⁴ is CH₃; or
R¹³ is CH₃ and R¹⁴ is H.

In some embodiments, the 3-(azetidin-2-ylmethoxy)pyridine has a structure selected from the group consisting of:

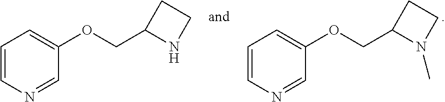

These compounds are known in the literature. The synthesis of these compounds has been reported in International Patent Application Publication No. WO2012/031220, previously incorporated by reference herein.

In some embodiments, the 3-(azetidin-2-ylmethoxy)pyridine has a structure selected from the group consisting of:

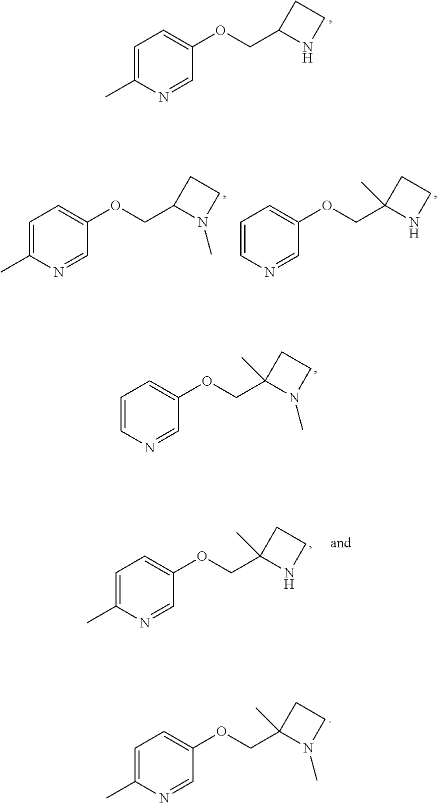

Such compounds are either known, or may be readily prepared according to adaptations of methods utilized for preparation of related the 3-(azetidin-2-ylmethoxy)pyridine, and/or the 3-(azetidin-2-yl)pyridines and substituted 3-(1-methylpyrrolidin-2-yl)pyridines described herein above.

An optionally substituted 3-(azetidin-2-yl)pyridine or optionally substituted 3-(azetidin-2-ylmethoxy)pyridine as described herein may be present as a single enantiomer or as a mixture of enantiomers. In some embodiments, the optionally substituted 3-(azetidin-2-yl)pyridine or optionally substituted 3-(azetidin-2-ylmethoxy)pyridine is present in racemic form, meaning there are equal amounts of (R)- and (S)-enantiomers present. In some embodiments, the aerosol generating material comprises unequal amounts of (R)- and (S)-enantiomer (i.e., is enriched in either the (R)- or (S)-enantiomer). In some embodiments, the aerosol generating material predominantly comprises the optionally substituted 3-(azetidin-2-yl)pyridine or optionally substituted 3-(azetidin-2-ylmethoxy)pyridine in the (R)-configuration, for example, about 90% or more of the total quantity of optionally substituted 3-(azetidin-2-yl)pyridine or optionally substituted 3-(azetidin-2-ylmethoxy)pyridine present is in the (R)-configuration. In some embodiments, the aerosol generating material predominantly comprises the optionally substituted 3-(azetidin-2-yl)pyridine or optionally substituted 3-(azetidin-2-ylmethoxy)pyridine in the (S)-configuration, for example, about 90% or more of the total quantity of optionally substituted 3-(azetidin-2-yl)pyridine or optionally substituted 3-(azetidin-2-ylmethoxy)pyridine present is in the (S)-configuration. In some embodiments, the aerosol generating material comprises 95% or more of the (S)-configuration of the optionally substituted 3-(azetidin-2-yl)pyridine or optionally substituted 3-(azetidin-2-ylmethoxy)pyridine, based on the total amount of optionally substituted 3-(azetidin-2-yl)pyridine or optionally substituted 3-(azetidin-2-ylmethoxy)pyridine present.

In some embodiments, the optionally substituted 3-(azetidin-2-yl)pyridine is non-racemic, and has one of the following structures:

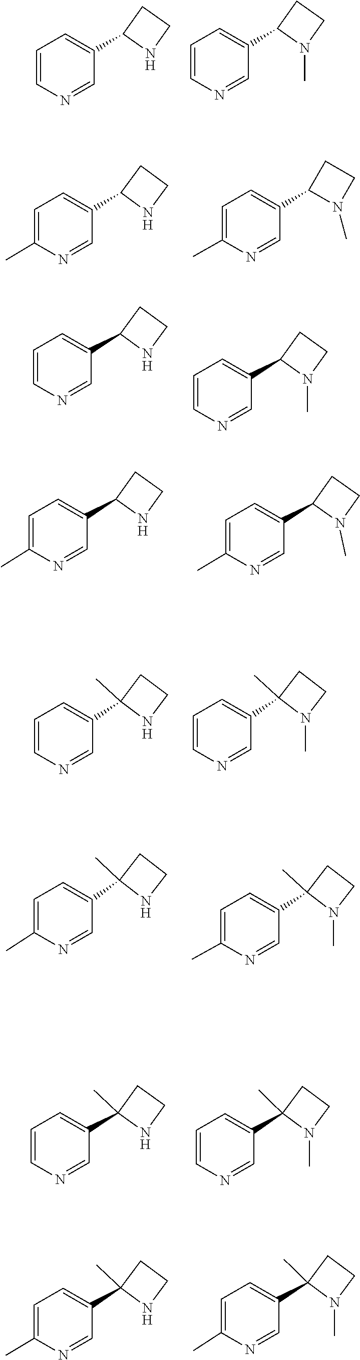

In some embodiments, the optionally substituted 3-(azetidin-2-ylmethoxy)pyridine is non-racemic, and has one of the following structures:

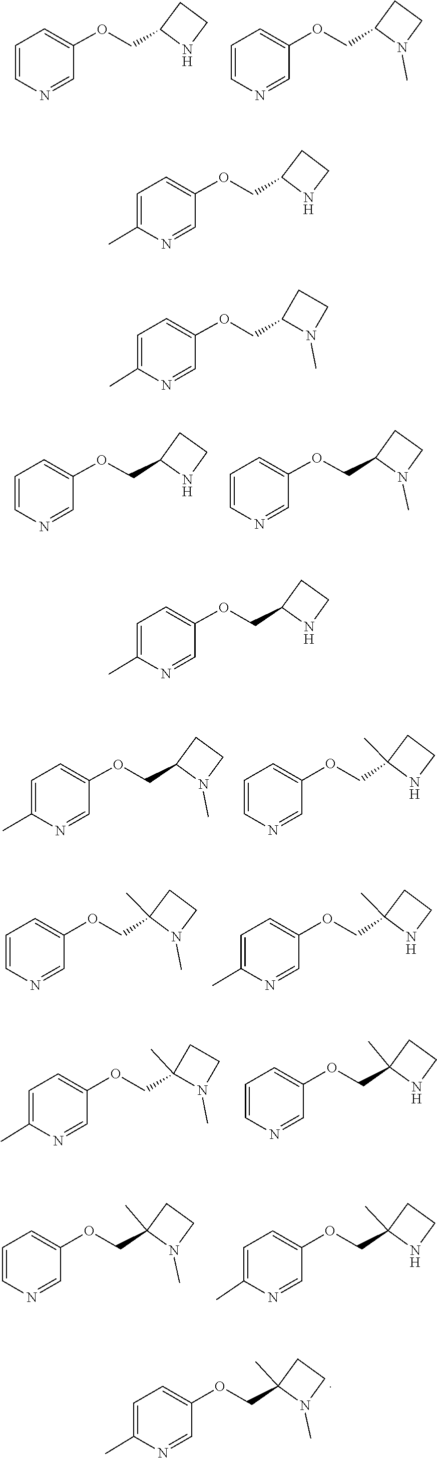

Such single enantiomer or enantiomerically enriched compounds may be obtained through classical resolution techniques using salt formation with chiral acids to form diastereomeric salts separable by crystallization. Suitable chiral acids include, but are not limited to, (R)- or (S)-dibenzoyl tartaric acid, di-p-toluoyl tartaric acid, or di-p-anisolyl tartaric acid; (R)- or (S)-mandelic acid, and (R)- or (S)-10-camphorsulfonic acid. Alternatively, one of skill in the art will recognize opportunities for chiral syntheses using either commercially available starting materials with established chiral centers or through the use of chiral auxiliary chemistries. For example, preparation of the 2S,4R enantiomer of 3-(1,4-dimethylpyrrolidin-2-yl)pyridine has been reported in, for example, U.S. Pat. No. 4,332,945, incorporated herein by reference with respect to syntheses of chiral nicotine analogs.

The pharmacology of certain 3-(azetidin-2-yl)pyridines and 3-(azetidin-2-ylmethoxy)pyridines has been previously disclosed, for example, in the references cited herein with respect to synthesis of such compounds. Generally, these compounds exhibit high affinity for one or more subtypes of nicotinic acetylcholine receptors, particularly the α4β2subtype. The overall pharmacological profiles have been shown to be or are expected to be comparable to that of nicotine.

The quantity of optionally substituted 3-(azetidin-2-yl)pyridine or 3-(azetidin-2-ylmethoxy)pyridine present in the aerosol generating material may vary. Typically, the optionally substituted 3-(azetidin-2-yl)pyridine or 3-(azetidin-2-ylmethoxy)pyridine, calculated as the free base, is present in a concentration of at least about 0.001% by weight of the aerosol generating material, such as in a range from about 0.01% to about 10%. In some embodiments, the optionally substituted 3-(azetidin-2-yl)pyridine or 3-(azetidin-2-ylmethoxy)pyridine is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the optionally substituted 3-(azetidin-2-yl)pyridine or 3-(azetidin-2-ylmethoxy)pyridine is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular optionally substituted 3-(azetidin-2-yl)pyridine or 3-(azetidin-2-ylmethoxy)pyridine present in the aerosol generating material may vary based on the potency of the compound, the aerosol generating material matrix, and the desired physiological effect for the aerosol generating material.

In some embodiments, the amount of optionally substituted 3-(azetidin-2-yl)pyridine or 3-(azetidin-2-ylmethoxy)pyridine in the aerosol generating material is determined by potency relative to nicotine. For example, in some embodiments, the amount of optionally substituted 3-(azetidin-2-yl)pyridine or 3-(azetidin-2-ylmethoxy)pyridine is based on the potency factor as described above for substituted 3-(1-methylpyrrolidin-2-yl)pyridines. In some embodiments, an optionally substituted 3-(azetidin-2-yl)pyridine or 3-(azetidin-2-ylmethoxy)pyridine of the disclosure has a potency factor from about 0.1 to about 30, such as from about 2 to about 30.

In some embodiments, the amount of optionally substituted 3-(azetidin-2-yl)pyridine or 3-(azetidin-2-ylmethoxy)pyridine in the aerosol generating material is 1 nicotine equivalent. Accordingly, in some embodiments, 1 nicotine equivalent of optionally substituted 3-(azetidin-2-yl)pyridine or 3-(azetidin-2-ylmethoxy)pyridine is an amount by weight from about 10 to about 0.03 times that of nicotine. In some embodiments, 1 nicotine equivalent of optionally substituted 3-(azetidin-2-yl)pyridine or 3-(azetidin-2-ylmethoxy)pyridine is an amount by weight from about 0.5 to about 0.03 times that of nicotine.

For example, a product comprising 2 mg of nicotine, when the nicotine is replaced by an optionally substituted 3-(azetidin-2-yl)pyridine or 3-(azetidin-2-ylmethoxy)pyridine of the disclosure, may include from about 0.06 mg to about 1 mg of the optionally substituted 3-(azetidin-2-yl)pyridine or 3-(azetidin-2-ylmethoxy)pyridine. Similarly, a product comprising 20 mg of nicotine, when the nicotine is replaced by an optionally substituted 3-(azetidin-2-yl)pyridine or 3-(azetidin-2-ylmethoxy)pyridine, may contain from about 0.6 mg to about 100 mg of the optionally substituted 3-(azetidin-2-yl)pyridine or 3-(azetidin-2-ylmethoxy)pyridine.

In some embodiments, the optionally substituted 3-(azetidin-2-yl)pyridine has the structure:

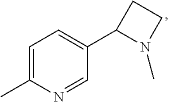

having a potency factor of about 30, meaning that in some embodiments, the amount of optionally substituted 3-(azetidin-2-yl)pyridine present may be about 3% of the amount of nicotine required to achieve the same effect.

In some embodiments, the optionally substituted 3-(azetidin-2-ylmethoxy)pyridine has the structure:

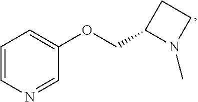

having a potency factor of about 3, meaning that in some embodiments, the amount of 3-(azetidin-2-ylmethoxy)pyridine present may be about 33% of the amount of nicotine required to achieve the same effect.

The optionally substituted 3-(azetidin-2-yl)pyridine or 3-(azetidin-2-ylmethoxy)pyridine may be present in the aerosol generating material as the free base, as a salt with a suitable acid, or in the form of an ion pair with an organic acid. Each of these forms is described further herein below.

### Other Active Agents

In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine of the present disclosure is replaced with, or combined with, other active agents that provide the same general pharmacological profile and/or physiological effects of nicotine. Certain of these active agents may be equipotent or even more potent than nicotine with respect to binding affinity to the nicotinic acetylcholine receptor and are expected to preserve the pharmacological effects of nicotine in vivo. Without wishing to be bound by any particular theory, in some embodiments, these compounds are believed to provide the general pharmacological profile and physiological effects of nicotine while offering the potential for one or more of greater potency, reduced product consumption, more rapid and/or complete absorption, and the like.

Example active agents of this type include, without limitation, cytisine, varenicline, acetylcholine, choline, epibatidine, lobeline, analogs thereof, or combinations thereof. Suitable analogs include any of the above-noted compounds having one or more substituents on any of the carbon atoms thereof, with example substituents including alkyl (e.g., C₁-C₃ alkyl), alkoxy, cycloalkyl, alkenyl, alkynyl, aryl, alkylaryl, amino, halogen, and cyano.

In some embodiments, the other active agents is cytisine or an analog thereof. Cytisine is a naturally occurring alkaloid present in certain plant genera, such as Laburnum and Cytisus of the family Fabaceae. Cytisine (CAS Registry No. 485-35-8) has the structure:

[symbol]

Cytisine is commercially available and has been utilized in post-Soviet states for more than 40 years as an aid to smoking cessation under the brand name Tabex (Sopharma AD). Cytisine is a partial agonist of the α₄β₂ nicotinic acetylcholine receptor.

In some embodiments, the other active agents is varenicline or an analog thereof. Varenicline is commercially available as Chantix® (Pfizer) and is a medication used as an aid for smoking cessation. Varenicline (CAS Registry No. 249296-44-4) has the structure:

[symbol]

Like cytisine, varenicline is a partial agonist of the α₄β₂ nicotinic acetylcholine receptor.

The quantity of the other active agent present in the aerosol generating material may vary. Typically, the other active agent, calculated as the free base, is present in a concentration of at least about 0.001% by weight of the aerosol generating material, such as in a range from about 0.01% to about 10%. In some embodiments, the other active agent is present in a concentration from about 0.05% w/w to about 5% by weight, such as, e.g., from about from about 0.05% w/w. about 0.1% w/w, about 0.2%, about 0.3%, about 0.4%, about 0.5% about 0.6%, about 0.7%, about 0.8%, or about 0.9%, to about 1%, about 2%, about 3%, about 4%, or about 5% by weight, calculated as the free base and based on the total weight of the aerosol generating material. In some embodiments, the other active agent is present in a concentration from about 0.05% w/w to about 4% by weight, such as, e.g., from about 0.05% w/w to about 3.5%, from about 0.07% to about 2.5%, from about 0.1% to about 2.0%, from about 0.1% to about 1.5%, or from about 0.1% to about 1% by weight, calculated as the free base and based on the total weight of the aerosol generating material. One of skill in the art will recognize that the amount of any particular other active agent present in the aerosol generating material may vary based on the potency of the compound, the aerosol generating material matrix, and the desired physiological effect for the aerosol generating material.

The other active agent may be present in the aerosol generating material as the free base, as a salt with a suitable acid, or in the form of an ion pair with an organic acid. Each of these forms is described further herein below.

### Free Base

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent exhibits sufficient stability, aqueous solubility, and oral bioavailability such that the free base is suitable for inclusion in the aerosol generating material. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine or other active agent is present substantially or completely as the free base. In such embodiments, one of skill in the art will recognize that the aerosol generating material is substantially free of acidic components. By “substantially free” it is meant that no acidic component (e.g., inorganic acid, organic acid, or acids capable of salt has been intentionally added, beyond trace amounts that may be present e.g., as an impurity in another component, or small amounts which may be present in certain flavor packages. For example, some embodiments can have less than 0.001% by weight of any acid component, or less than 0.0001%, or even 0% by weight of any acid component, based on the total weight of the aerosol generating material. In some embodiments, the aerosol generating material is completely free of any acid component (i.e., having 0% or having an amount below the limit of detection). In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the free base form and is adsorbed in a carrier such as a microcrystalline cellulose material to form an adsorption complex.

### Salt

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be employed in the form of a salt. A “salt” of such compounds is a form characterized by interaction between the said compound in ionic form and a coformer in ionic form (e.g., an acid) via the transfer of one or more protons from the coformer donor to the compound acceptor. The structure of substituted 3-(1-methylpyrrolidin-2-yl)pyridines, optionally substituted 3-(azetidin-2-yl)pyridines, and optionally substituted 3-(azetidin-2-ylmethoxy)pyridines as disclosed herein are such that they comprise two nitrogen atoms that are capable of accepting protons from a coformer and, accordingly, can be present in non-protonated, mono-protonated, and/or di-protonated form in a given sample. Salts of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent can be provided using the types of ingredients and techniques set forth for nicotine in U.S. Pat. No. 2,033,909 to Cox et al. and Perfetti, Beitrage Tabakforschung Int., 12: 43-54 (1983), which are incorporated herein by reference Suitable salts are generally water soluble. Suitable acids for formation of salts (mono- and di-) include, but are not limited to, acetic acid, adipic acid, ascorbic acid, capric acid, citric acid, D-glucuronic acid, D-gluconic acid, lactic acid, galactaric acid, hippuric acid, hydrochloric acid, L-aspartic acid, L-glutamic acid, L-glutaric acid, glycerophosphoric acid, glycolic acid, lauric acid, DL-malic acid, L-malic acid; tartaric acid, palmitic acid, phosphoric acid, sebacic acid, stearic acid, succinic acid, sulfuric acid, thiocyanic acid, (+)-camphoric acid, 1,5-naphthalenedisulfonic acid, 1-hydroxy-2-naphthoic, 2,5-dihydroxybenzoic acid, benzenesulfonic acid, benzoic acid, caprylic acid, cyclamic acid, ethanesulfonic acid, fumaric acid, D-glucoheptonic acid, 4-hydroxybenzoic acid, isobutyric acid, ketoglutaric acid, 2-ketobutyric acid, lactobionic acid, maleic acid, malonic acid, methanesulfonic acid, naphthalene-2-sulfonic acid, nicotinic acid, oleic acid, orotic acid, oxalic acid, pamoic acid, pivalic acid, propionic acid, L-pyroglutamic acid, p-toluenesulfonic acid, (1S)-camphor-10-sulfonic acid, 4-acetamidobenzoic acid, 4-aminosalicylic acid, N-acetyl-4-aminosalicylic acid, caproic acid, dichloroacetic acid, hydrobromic acid, DL-mandelic acid, L-mandelic acid, nitric acid, formic acid, salicylic acid, cinnamic acid, undecylenic acid, isothionic acid, lauric acid, 2-hydroxybenzoic acid, trans-2-hexanoic acid, trimesic acid, 5-nitroisophthalic acid and zinc chloride monohydrate (forming a hydrated zinc chloride complex salt). In some embodiments, the salt is with an organic acid as described herein below.

In some embodiments, a hydrophilic acid is chosen so as to increase water solubility and/or decrease lipophilicity of the salt. Lipophilicity of a salt of a compound as disclosed herein can also be expressed as log D, which is the logarithm of the distribution coefficient, a measure of the pH-dependent differential solubility between an octanol phase and an aqueous phase of all species (ionized and un-ionized) in an octanol/aqueous system, represented by the formula:

$$log {D}_{oct / wat} = log ( \frac{{[ solute ]}_{octanol}}{{[ solute ]}_{water}^{inonized} + {[ solute ]}_{water}^{neutral}} ) .$$

Log D is a commonly used descriptor for the lipophilicity of ionizable compounds. Log D values can be calculated using commercial software or may be determined experimentally in a similar manner to log P but instead of using water, the aqueous phase is adjusted to a specific pH using a buffer. Log D is pH dependent and therefore requires that the pH at which the log D was measured be specified.

When the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt, it is generally preferred that the salt have a relatively low log D, indicative of good water solubility. Without wishing to be bound by theory, it is believed that highly water-soluble salt forms may exhibit a high rate of dissolution, which may be favorable in certain embodiments. Accordingly, in some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent salt has a log D from about −1.0 to about 3 at a pH in a range from about 3 to about 11, such as from about −0.5 to about 2, about −0.3 to about 1, or about −0.1 to about 0.

In some embodiments, the selection of acid used to make a salt is performed on the basis of sensory effects of the salt, such as taste. Surprisingly, according to the present disclosure, it has been found that salts of certain organic acids, such as galactaric acid, offer a better taste sensation relative to salts of acids such as tartaric or phthalic acids.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt with tartaric acid, succinic acid, orotic acid, fumaric acid, pyroglutamic acid, or galactaric acid. In some embodiments, the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a salt with succinic acid or galactaric acid.

The stoichiometry of the salts as described herein can vary. For example, in some embodiments, the stoichiometry can range from about 5:1 to about 1:5 molar equivalents of compound:acid, including any whole or fractional value in between. In some embodiments, the molar ratio of compound to acid is about 2:1, about 1:1, or about 1:2. In some embodiments, the compound is present in the form of a bitartrate salt. Hydrates and other solvates of salts are further contemplated herein.

The salts as described herein can, in some embodiments, exist in various polymorphic and pseudopolymorphic forms. Polymorphism is the ability of a crystalline material to exist in more than one form or crystal structure. Polymorphism can result, e.g., from the existence of different crystal packing structures (packing polymorphism) or from the existence of different conformers of the same molecule (conformational polymorphism). Pseudopolymorphism is the result of hydration or solvation of a material and is also referred to as solvomorphism.

### Organic Acid

In some embodiments, the aerosol generating material comprises an organic acid. In some embodiments, the organic acid is a carboxylic acid or a sulfonic acid. The carboxylic acid or sulfonic acid functional group may be attached to any alkyl, cycloalkyl, heterocycloalkyl, aryl, or heteroaryl group having, for example, from one to twenty carbon atoms (C₁-C₂₀). In some embodiments, the organic acid is an alkyl, cycloalkyl, heterocycloalkyl, aryl, or heteroaryl carboxylic or sulfonic acid.

As used herein, “alkyl” refers to any straight chain or branched chain hydrocarbon. The alkyl group may be saturated (i.e., having all sp³ carbon atoms), or may be unsaturated (i.e., having at least one site of unsaturation). As used herein, the term “unsaturated” refers to the presence of a carbon-carbon, sp² double bond in one or more positions within the alkyl group. Unsaturated alkyl groups may be mono- or polyunsaturated. Representative straight chain alkyl groups include, but are not limited to, methyl, ethyl, n-propyl, n-butyl, n-pentyl, and n-hexyl. Branched chain alkyl groups include, but are not limited to, isopropyl, sec-butyl, isobutyl, tert-butyl, isopentyl, and 2-methylbutyl. Representative unsaturated alkyl groups include, but are not limited to, ethylene or vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkyl group can be unsubstituted or substituted.

“Cycloalkyl” as used herein refers to a carbocyclic group, which may be mono- or bicyclic. Cycloalkyl groups include rings having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted, and may include one or more sites of unsaturation (e.g., cyclopentenyl or cyclohexenyl).

The term “aryl” as used herein refers to a carbocyclic aromatic group. Examples of aryl groups include, but are not limited to, phenyl and naphthyl. An aryl group can be unsubstituted or substituted.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl group comprises up to 20 carbon atoms and from 1 to 3 heteroatoms selected from N, O, and S. A heteroaryl or heterocycloalkyl may be a monocycle having 3 to 7 ring members (for example, 2 to 6 carbon atoms and 1 to 3 heteroatoms selected from N, O, and S) or a bicycle having 7 to 10 ring members (for example, 4 to 9 carbon atoms and 1 to 3 heteroatoms selected from N, O, and S), for example: a bicyclo[4,5], [5,5], [5,6], or [6,6] system. Examples of heteroaryl groups include by way of example and not limitation, pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl. Examples of heterocycloalkyls include by way of example and not limitation, dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl. Heteroaryl and heterocycloalkyl groups can be unsubstituted or substituted.

“Substituted” as used herein and as applied to any of the above alkyl, aryl, cycloalkyl, heteroaryl, heterocyclyl, means that one or more hydrogen atoms are each independently replaced with a substituent. Typical substituents include, but are not limited to, —Cl, Br, F, alkyl, —OH, —OCH₃, NH₂, —NHCH₃, —N(CH₃)₂, —CN, —NC(═O)CH₃, —C(═O)—, —C(═O)NH₂, and —C(═O)N(CH₃)₂. Wherever a group is described as “optionally substituted,” that group can be substituted with one or more of the above substituents, independently selected for each occasion. In some embodiments, the substituent may be one or more methyl groups or one or more hydroxyl groups.

In some embodiments, the organic acid is an alkyl carboxylic acid. Non-limiting examples of alkyl carboxylic acids include formic acid, acetic acid, propionic acid, butyric acid, valeric acid, caproic acid, heptanoic acid, octanoic acid, nonanoic acid, decanoic acid, undecanoic acid, dodecanoic acid, stearic acid, oleic acid, linoleic acid, linolenic acid, and the like.

In some embodiments, the organic acid is an alkyl sulfonic acid. Non-limiting examples of alkyl sulfonic acids include propanesulfonic acid, heptanesulfonic acid, and octanesulfonic acid.

In some embodiments, the alkyl carboxylic or sulfonic acid is substituted with one or more hydroxyl groups. Non-limiting examples include glycolic acid, 4-hydroxybutyric acid, and lactic acid.

In some embodiments, an organic acid may include more than one carboxylic acid group or more than one sulfonic acid group (e.g., two, three, or more carboxylic acid groups). Non-limiting examples include oxalic acid, fumaric acid, maleic acid, and glutaric acid. In organic acids containing multiple carboxylic acids (e.g., from two to four carboxylic acid groups), one or more of the carboxylic acid groups may be esterified. Non-limiting examples include succinic acid monoethyl ester, monomethyl fumarate, monomethyl or dimethyl citrate, and the like.

In some embodiments, the organic acid may include more than one carboxylic acid group and one or more hydroxyl groups. Non-limiting examples of such acids include tartaric acid, citric acid, and the like.

In some embodiments, the organic acid is an aryl carboxylic acid or an aryl sulfonic acid. Non-limiting examples of aryl carboxylic and sulfonic acids include benzoic acid, toluic acids, salicylic acid, benzenesulfonic acid, and p-toluenesulfonic acid.

Further non-limiting examples of organic acids which may be useful in certain embodiments include 2-(4-isobutylphenyl)propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, cyclamic acid, dodecylsulfuric acid, ethane-1,2-disulfonic acid, ethanesulfonic acid, furoic acid, galactaric acid, gentisic acid, glucoheptonic acid, gluconic acid, glucuronic acid, glutamic acid, glycerophosphoric acid, glycolic acid, hippuric acid, isobutyric acid, isovaleric acid, lactobionic acid, lauric acid, levulinic acid, malic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, and undecylenic acid. Examples of suitable acids include, but are not limited to, the list of organic acids in Table 1.

TABLE 1
Non-limiting examples of suitable organic acids
Acid Name

benzoic acid
phenylacetic
p-toluic acid
ethyl benzoic acid
isopropyl benzoic acid
4-phenylbutyric
2-(4-Isobutylphenyl)propanoic acid
2-napthoxyacetic acid
napthylacetic acid
heptanoic acid
octanoic acid
nonanoic acid
decanoic acid
9-deceneoic acid
2-deceneoic acid
10-undecenoic acid
dodecandioic acid
dodecanoic acid
myristic acid
palmitic acid
stearic acid
cyclohexanebutanoic acid
1-heptanesulfonic acid
1-octanesulfonic acid
1-nonanesulfonic acid
monooctyl succinate
tocopherol succinate
monomenthyl succinate
monomenthyl glutarate
norbixin ((2E,4E,6E,8E,10E,12E,14E,16E,18E)-4,8,13,17-
tetramethylicosa-2,4,6,8,10,12,14,16,18-nonaenedioic acid)
bixin ((2E,4E,6E,8E,10E,12E,14E,16Z,18E)-20-methoxy-
4,8,13,17-tetramethyl-20-oxoicosa-2,4,6,8,10,12,14,16,18-
nonaenoic acid)

The selection of organic acid may depend on certain properties. For example, an organic acid should be one recognized as safe for human consumption, and which has acceptable flavor, odor, volatility, stability, and the like. Determination of appropriate organic acids is within the purview of one of skill in the art. In some embodiments, the acid is benzoic acid. In some embodiments, the acid is levulinic acid. In some embodiments, the acid is lactic acid. In some embodiments, the acid is one or more of benzoic acid, lactic acid, and levulinic acid.

In some embodiments, more than one organic acid may be present. For example, the aerosol modifying agent may comprise two, or three, or four, or more organic acids. Accordingly, reference herein to “an organic acid” contemplates mixtures of two or more organic acids. The relative amounts of the multiple organic acids may vary. For example, an aerosol modifying agent may comprise equal amounts of two, or three, or more organic acids, or may comprise different relative amounts. Without wishing to be bound by theory, it is believed that a combination of different organic acids may provide a concentration of any single organic acid in the aerosol generating material which remains below the threshold which would be found objectionable from a sensory perspective.

The amount of organic acid present in the article (e.g., in the aerosol generating material and/or the body of material), relative to the substituted 3-(1-methylpyrrolidin-2-yl)pyridine, 3-(azetidin-2-yl)pyridine, or 3-(azetidin-2-ylmethoxy)pyridine (e.g., 2-methyl-5-(1-methylpyrrolidin-2-yl)pyridine), may vary. In some embodiments, the aerosol generating material comprises from about 0.05, about 0.1, about 1, about 1.5, about 2, or about 5, to about 10, about 15, or about 20 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, calculated as the free base of the 3-(1-methylpyrrolidin-2-yl)pyridine, 3-(azetidin-2-yl)pyridine, or 3-(azetidin-2-ylmethoxy)pyridine.

In some embodiments, the aerosol generating material comprises from about 2 to about 10, or from about 2 to about 5 molar equivalents of the organic acid relative to the substituted 3-(1-methylpyrrolidin-2-yl)pyridine, 3-(azetidin-2-yl)pyridine, or 3-(azetidin-2-ylmethoxy)pyridine, on a free-base basis. In some embodiments, the organic acid is present in a molar ratio with the substituted 3-(1-methylpyrrolidin-2-yl)pyridine, 3-(azetidin-2-yl)pyridine, or 3-(azetidin-2-ylmethoxy)pyridine from about 2, about 3, about 4, or about 5, to about 6, about 7, about 8, about 9, or about 10. In embodiments wherein more than one organic acid it is to be understood that such molar ratios reflect the totality of the organic acids present.

In some embodiments, the aerosol generating material comprises one or more of the organic acids described herein above. The substituted 3-(1-methylpyrrolidin-2-yl)pyridine, 3-(azetidin-2-yl)pyridine, or 3-(azetidin-2-ylmethoxy)pyridine and the one or more organic acids may be present independently, in the form of a salt, or as a cocrystal. In some embodiments, the aerosol generating material comprises one or more of lactic acid, levulinic acid, benzoic acid, succinic acid, galactaric acid, orotic acid, fumaric acid, pyroglutamic acid, and tartaric acid, and at least a portion of said acid is present in the form of a salt with the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a lactic acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a levulinic acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a galactaric acid salt of the substituted 3-(1-methylpyrrolidin-2-yl)pyridine, 3-(azetidin-2-yl)pyridine, or 3-(azetidin-2-ylmethoxy)pyridine.

In some embodiments, the aerosol generating material comprises a tartaric acid salt of the substituted 3-(1-methylpyrrolidin-2-yl)pyridine, 3-(azetidin-2-yl)pyridine, or 3-(azetidin-2-ylmethoxy)pyridine.

The stoichiometry of such lactate, levulinate, tartrate, and galactarate salts may vary. Accordingly, the molar ratio of lactic, levulinic, tartaric, or galactaric acid to substituted 3-(1-methylpyrrolidin-2-yl)pyridine, 3-(azetidin-2-yl)pyridine, or 3-(azetidin-2-ylmethoxy)pyridine may be in a range from about 3:1 to about 1:3, including any whole or fractional value in between, such as for example about 2:1, about 1.5:1, about 1:1, about 1:1.5, or about 1:2.

In some embodiments, the aerosol generating material may be substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection).

### Resin Complex

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a polymer complex, where the compound is bound to an acidic polymer. The polymer of such a complex can be any polymer (including homopolymers or all types of copolymers) with acidic functionalities, e.g., a polymeric cation exchange resin. In some embodiments, the polymer comprises acidic sites that can be classified as strongly acidic, weakly acidic, or of intermediate acidity (depending, e.g., on the strength of the acid from which they are derived). In some embodiments, the polymer comprises weakly acidic sites and can be referred to as a weakly acidic cation exchange resin. Non-limiting examples of acidic sites include, e.g., carboxylic acids, sulfonic acids, phosphonous acids, phosphonic acids, phosphoric acids, iminodiacetic acids, and phenolic groups (e.g., as disclosed in Adams et al., J. Soc. Chem. Ind. 54, IT (1935), which is incorporated herein by reference). Suitable polymers include, but are not limited to, addition polymers of styrene and divinylbenzene, divinylbenzene and methacrylic acid, divinylbenzene and acrylic acid, phenolic resins, or cellulose, dextran or pectin cross-linked with, e.g., epichlorohydrin. In some embodiments, the polymer comprises cross-linked moieties. Various acidic ion-exchange resins which are known in the art and are suitable for formation of complexes, include, but are not limited to, polymethacrylic acid resins such as DuPont™ Amberlite™ IRP64, DuPont™ Amberlite™ IRP69, Purolite™ C115HMR, Doshion™ P551, and polyacrylic carbomers, such as Carbopol 974P. See, for example, U.S. Pat. No. 3,901,248 to Lichtneckert et al., which is incorporated herein by reference. In some embodiments, when the substituted 3-(1-methylpyrrolidin-2-yl)pyridine, optionally substituted 3-(azetidin-2-yl)pyridine, or optionally substituted 3-(azetidin-2-ylmethoxy)pyridine is present in the form of a polymer complex, the aerosol generating material further comprises a divalent metal buffer, such as a calcium or magnesium salt (e.g., carbonate, bicarbonate, oxide, acetate, or the like).

### Cocrystal

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent may be present in the form of a co-crystal with at least one other component (“coformer”), both in neutral form. Specifically, as defined in a US FDA industry guidance document, a co-crystal is a solid that is a crystalline material composed of two or more molecules in the same crystal lattice, where the components are in a neutral state and interact via nonionic interactions. See U.S. Department of Health and Human Services, Food and Drug Administration, Guidance for Industry: Regulatory Classification of Pharmaceutical Co-Crystals (April 2013), which is incorporated herein by reference. This form is different and distinct from both salts and ion pairs, each described herein. Specifically, co-crystals can generally be distinguished from salts (and ion pairs) by the absence of a proton transfer between the components (i.e., a substituted 3-(1-methylpyrrolidin-2-yl)pyridine and the one or more coformers) in a co-crystal. The crystalline structure of the co-crystal is generally held together by freely reversible, non-covalent interactions. Co-crystals typically comprise the substituted 3-(1-methylpyrrolidin-2-yl)pyridine, optionally substituted 3-(azetidin-2-yl)pyridine, or optionally substituted 3-(azetidin-2-ylmethoxy)pyridine and coformer in a defined stoichiometric ratio. In some embodiments, co-crystals can encompass hydrates, solvates, and clathrates. Co-crystals can comprise the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in combination with an organic and/or an inorganic coformer.

Examples of suitable coformers include, but are not limited to, acetamidobenzoic acid, L-proline, tromethamine, urea, xylitol, caffeine, glycine/glycine anhydride, vanillin, methyl 4-hydroxybenzoate(methylparaben), succinimide, L-alanine, mannitol, L-phenylalanine, saccharin, propylparaben, N-methylglucamine, L-tyrosine, gentisic acid, sorbic acid, benzoic acid, L-methionine, maltol, L-lysine, tromethamine, nicotinamide, isonicotinamide, phenylalanine, benzoquinone, terephthalaldehyde, 4-hydroxybenzoic acid, pyruvic acid, 1-hydroxy-2-naphthoic acid, 4-aminobenzoic acid, vanillic acid, ethyl vanillin, isonicotinic acid, gallic acid, menthol (e.g., racemic menthol or (−)-menthol), paracetamol, aspirin, ibuprofen, naproxen, ketoprofen, flurbiprofen, glucose, serine, malic acid, acetamide, sulfacetamide, benzoic acid, creatine, 2-hydroxyethanesulfonic acid, clofibric acid, taurine (tauric acid), iproniazid, L-histidine, L-arginine, L-asparagine, glutamine, L-cysteine, alanine, valine, isoleucine, leucine, morpholine, threonine, N-methylglucamine, 3-hydroxy-2-oxopropionic acid; 2-oxobutyric acid (2-ketobutyric acid), 3-methyl-2-oxobutanoic acid; 3-methyl-2-oxopentanoic acid; 4-methyl-2-oxopentanoic acid; and 2-oxopentanedioic acid, 2-oxo-3-phenylpropionic acid; 5-oxooctanoic acid; and 5-oxodecanoic acid, aldonic acids (e.g., glyceric acid, xylonic acid, gluconic acid, and ascorbic acid), ulosonic acids (e.g., neuraminic acid and ketodeoxyoctulosonic acid), uronic acids (e.g., glucuronic acid, galacturonic acid, and iduronic acid), aldaric acids (e.g., tartaric acid, meso-galactaric acid/mucic acid, and D-glucaric acid/saccharic acid), galactaric acid), and polyfunctional aromatic acids.

In some embodiments, the conformer is a polyfunctional aromatic acid. Polyfunctional aromatic acids often comprise a substituted or unsubstituted phenyl group as the aromatic component, but can alternatively comprise another aromatic moiety, e.g., pyridine, pyrazine, imidazole, pyrazole, oxazole, thiophene, naphthalene, anthracene, and phenanthrene. Substituents on the optionally substituted aromatic acids may be any type of substituent, including, but not limited to, halo (e.g., Cl, F, Br, and I); alkyl, halogenated alkyl (e.g., CF₃, 2-Br-ethyl, CH₂F, CH₂Cl, CH₂CF₃, or CF₂CF₃); alkenyl, hydroxyl; amino; carboxylate; carboxamido; alkylamino; arylamino; alkoxy; aryloxy; nitro; azido; cyano; thio; sulfonic acid; sulfate; phosphonic acid; phosphate; and phosphonate groups. Example polyfunctional aromatic acids can be, for example:
substituted and unsubstituted aromatic dicarboxylic acids (e.g., 1,2-benzenedicarboxylic acid (phthalic acid), 1,3-benzenedicarboxylic acid (isophthalic acid), 1,4-benzenedicarboxylic acid (terephthalic acid), 2-iodo-1,3-benzenedicarboxylic acid, 2-hydroxy-1,4-benzenedicarboxylic acid, 2-nitro-1,4-benzenedicarboxylic acid, 3-fluoro-1,2-benzenedicarboxylic acid, 3-amino-1,2-benzenedicarboxylic acid, 3-nitro-1,2-benzenedicarboxylic acid, 4-bromo-1,3-benzenedicarboxylic acid, 4-hydroxy-1,3-benzenedicarboxylic acid, 4-amino-1,2-benzenedicarboxylic acid, 4-nitro-1,2-benzenedicarboxylic acid, 4-sulfo-1,2-benzenedicarboxylic acid, 4-amino-1,3-benzenedicarboxylic acid, 5-bromo-1,3-benzenedicarboxylic acid, 5-hydroxy-1,3-benzenedicarboxylic acid, 5-amino-1,3-benzenedicarboxylic acid, 5-nitro-1,3-benzenedicarboxylic acid, 5-ethynyl-1,3-benzenedicarboxylic acid, 5-cyano-1,3-benzenedicarboxylic acid, 5-nitro-1,3-benzenedicarboxylic acid, 2,5-hydroxy-1,4-benzenedicarboxylic acid, and 2,3,5,6-tetrafluoro-1,4-benzenedicarboxylic acid;
substituted and unsubstituted hydroxybenzoic acids (e.g., 2-hydroxybenzoic acid (salicylic acid), 3-hydroxybenzoic acid, 4-hydroxybenzoic acid, 2-methyl-4-hydroxybenzoic acid, 3-tert-butyl-4-hydroxybenzoic acid, 4-ethoxy-2-hydroxybenzoic acid, 3-chloro-5-hydroxybenzoic acid, 5-chloro-2-hydroxybenzoic acid, 3-bromo-4-hydroxybenzoic acid, 3-bromo-5-hydroxybenzoic acid, 4-bromo-2-hydroxybenzoic acid, 5-bromo-2-hydroxybenzoic acid, 2-fluoro-5-hydroxybenzoic acid, 3-fluoro-4-hydroxybenzoic acid, 3-fluoro-2-hydroxybenzoic acid, 3-fluoro-5-hydroxybenzoic acid, 2-fluoro-6-hydroxybenzoic acid, 4-fluoro-3-hydroxybenzoic acid, 2-fluoro-4-hydroxybenzoic acid, 5-fluoro-2-hydroxybenzoic acid, 2-amino-3-hydroxybenzoic acid, 2-amino-5-hydroxybenzoic acid, 3-amino-2-hydroxybenzoic acid, 3-amino-4-hydroxybenzoic acid, 3-amino-5-hydroxybenzoic acid, 4-amino-2-hydroxybenzoic acid, 4-amino-3-hydroxybenzoic acid, 5-amino-2-hydroxybenzoic acid (mesalamine), 5-aminomethyl-2-hydroxybenzoic acid, 4-formyl-3-hydroxybenzoic acid, 3-formyl-4-hydroxybenzoic acid, 5-(acetylamino)-2-hydroxybenzoic acid), 4-nitro-2-hydroxybenzoic acid, 3,5-diethyl-4-hydroxybenzoic acid, 3,5-di-tert-butyl-4-hydroxybenzoic acid, 3,5-diisopropyl-2-hydroxybenzoic acid, 3,4-dimethoxy-4-hydroxybenzoic acid (syringic acid), 3,5-dichloro-2-hydroxybenzoic acid, 3,5-dichloro-4-hydroxybenzoic acid, 3,6-dichloro-2-hydroxybenzoic acid, 2,3-difluoro-4-hydroxybenzoic acid, 3,4-difluoro-2-hydroxybenzoic acid, 3,5-dibromo-2-hydroxybenzoic acid, 3,5-diiodo-2-hydroxybenzoic acid, 4-amino-5-chloro-2-hydroxybenzoic acid, 3,5-dinitro-2-hydroxybenzoic acid, 2,4,6-tribromo-2-hydroxybenzoic acid, 2,3,5,6-tetrafluoro-4-hydroxybenzoic acid, and 2,3,4,5-tetrafluoro-6-hydroxybenzoic acid);
substituted and unsubstituted dihydroxybenzoic acids (e.g., 2,3-dihydroxybenzoic acid (pyrocatechuic acid/hypogallic acid), 2,4-dihydroxybenzoic acid (β-resorcylic acid), 2,5-dihydroxybenzoic acid (gentisic acid/hydroquinonecarboxylic acid), 2,6-dihydroxybenzoic acid (γ-resorcylic acid), 3,4-dihydroxybenzoic acid (protocatechuic acid), 3,5-dihydroxybenzoic acid (α-resorcylic acid), 4-hydroxy-3-methoxybenzoic acid (vanillic acid), 6-methyl-2,4-dihydroxybenzoic acid (orsellenic acid), 4-bromo-3,5-dihydroxybenzoic acid, 5-bromo-2,4-dihydroxybenzoic acid, 5-bromo-3,4-dihydroxybenzoic acid, 6-carboxymethyl-2,3-dihydroxybenzoic acid, 3,5-dibromo-2,4-dihydroxybenzoic acid, 3,5-dichloro-2,6-dihydroxybenzoic acid, and 5-amino-3-chloro-2,4-dihydroxybenzoic acid);
substituted and unsubstituted trihydroxybenzoic acids (e.g., 2,3,4-trihydroxybenzoic acid, 2,4,5-trihydroxybenzoic acid, 2,4,6-trihydroxybenzoic acid (phloroglucinol carboxylic acid), and 3,4,5-trihydroxybenzoic acid (gallic acid));
substituted and unsubstituted aromatic tricarboxylic acids (e.g., 1,2,3-benzenetricarboxylic acid, 1,2,4-benzenetricarboxylic acid (trimellitic acid); and
substituted and unsubstituted aromatic tetracarboxylic acids (e.g., 1,2,3,4-benzenetetracarboxylic acid (mellophanic acid) and 1,2,4,5-benzenetetracarboxylic acid (pyromellitic acid). Further contemplated are various combinations of any of the foregoing acids.

In some embodiments, the coformer is L-malic acid, succinic acid, or a combination thereof. In some embodiments, the coformer is 1,1,6,6-tetraphenyl-2,4-hexidiyne-1,6-diol. In some embodiments, the coformer is di-iodotetrafluoro benzene, 4,4′-diiodooctafluorobiphenyl, or 1,4-bis(diphenylhydroxymethyl)benzene. In some embodiments, the coformer is orotic acid.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt co-crystal. A “salt co-crystal” is a type of hybrid structure with both salt and co-crystal characteristics. Typically, a substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent within a salt co-crystal is associated with at least two coformers (which may be the same or different), wherein one coformer is in ionic form (e.g., an acid) and transfers a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent, and wherein a second coformer does not transfer a proton to the substituted 3-(1-methylpyrrolidin-2-yl)pyridine, optionally substituted 3-(azetidin-2-yl)pyridine, optionally substituted 3-(azetidin-2-ylmethoxy)pyridine molecule, or other active agent. Suitable acids and coformers are generally those described herein above with respect to salts and co-crystals.

The stoichiometry of the co-crystals and salt co-crystals described herein can vary. For example, in certain embodiments, where two components are present, the stoichiometry can range in certain embodiments from about 5:1 to about 1:5 compound:coformer. Where more than one coformer is used to form a co-crystal or salt co-crystal, the ratios of the coformers with respect to both the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and to one another can also vary.

The co-crystals and salt co-crystals described herein can, in some embodiments, exist in various polymorphic and pseudopolymorphic forms, as well as solvates and hydrates.

In some embodiments, the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of a salt-co-crystal. In some embodiments, the salt-co-crystal is a bis-orotic acid salt-co-crystal. In some embodiments, the bis-orotic acid salt-co-crystal is a hemi-hydrate.

### Ion Pairing

In some embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is present in the form of an ion pair. Ion pairing describes the partial association of oppositely charged ions in relatively concentrated solutions to form distinct chemical species called ion pairs. The strength of the association (i.e., the ion pairing) depends on the electrostatic force of attraction between the positive and negative ions (e.g., a substituted 3-(1-methylpyrrolidin-2-yl)pyridine and the conjugate base of a suitable acid). By “conjugate base” is meant the base resulting from deprotonation of the corresponding acid (e.g., benzoate is the conjugate base of benzoic acid). In embodiments comprising ion pairing, on average, a certain population of these ion pairs exists at any given time, although the formation and dissociation of ion pairs is continuous. In some embodiments, in the aerosol generating material as disclosed herein, and/or upon oral use of said aerosol generating material (e.g., upon contact with saliva), the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent and the conjugate base of an acid exist at least partially in the form of an ion pair. Ion pairing is further described in, for example, International Patent Application Publication No. WO2021/050741 to Poole et al., and US Application Publication Nos. 2021/0068447 to Keller et al., 2023/0138306A1 to Zawadzki et al., and 2022/0346434 to Von Cosmos et al., each of which is incorporated herein by reference.

One of skill in the art will recognize that the extent of ion pairing in the disclosed aerosol generating material, both before and during use by the consumer, may vary based on, for example, pH, the nature of the acid, the concentration of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, the concentration of the acid or conjugate base of the acid present in the aerosol generating material, the moisture content of the aerosol generating material, the ionic strength of the aerosol generating material, and the like. One of skill in the art will also recognize that ion pairing is an equilibrium process influenced by the foregoing variables. Accordingly, quantification of the extent of ion pairing is difficult or impossible by calculation or direct observation. However, the presence of ion pairing may be demonstrated through surrogate measures, such as partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent between octanol and water, or by performing membrane permeation studies of aqueous solutions of, for example, the substituted 3-(1-methylpyrrolidin-2-yl)pyridine plus acids and/or their conjugate bases. An octanol-water partitioning favoring distribution of an ion pair into octanol is predictive of good absorption of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent through the oral mucosa. However, as described above, in some embodiments, the properties of the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent are such that no ion pairing is required, and accordingly, the aerosol generating material is substantially or completely free of any ion pairing. By “substantially free” it is meant that no measurable degree of ion pairing is present.

In embodiments where ion pairing is desired, the aerosol generating material comprises an organic acid, an alkali metal salt of an organic acid, or both. In such embodiments, at least a portion of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is associated with at least a portion of the organic acid, the alkali metal salt thereof, or a combination thereof in the form an ion pair. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H) or sulfonic acids (—SO₂OH). As used herein, reference to organic acid means an organic acid that is intentionally added. In this regard, an organic acid may be intentionally added as a specific aerosol generating material ingredient as opposed to merely being inherently present as a component of another aerosol generating material ingredient (e.g., the small amount of organic acid which may inherently be present in a aerosol generating material ingredient). For the avoidance of doubt, reference herein to an “organic acid” is intended to distinguish the acid present in ion paired forms over the acid which may be present in salts, co-crystal, and salt co-crystals. While one of skill in the art will recognize that certain organic acids suitable for formation of ion pairs overlap with those identified as suitable for salt or co-crystal formation, it is to be understood that the particular acid used for each of salts, co-crystals, and ion pairs are to be selected specifically for each such embodiment, and reference herein to an organic acid is specific to acids suitable for ion pairing. Accordingly, the presence in the aerosol generating material of an organic acid as defined below is to be interpreted solely with respect to ion pairing, even if such organic acid is also suitable for salt formation or co-crystal formation, and the presence of such an organic acid does not imply that a salt or co-crystal is present unless explicitly identified. Further, in embodiments where there is no ion pairing intended, the aerosol generating material may be characterized as substantially or completely free of organic acids (i.e., having less than 0.001% by weight of organic acid, or less than 0.0001%, or even 0% by weight of organic acid, based on the total weight of the aerosol generating material, or as having an amount of organic acid below the limit of detection). This is not to be interpreted as meaning that the aerosol generating material is substantially or completely free of substituted 3-(1-methylpyrrolidin-2-yl)pyridine salts or substituted 3-(1-methylpyrrolidin-2-yl)pyridine co-crystals unless explicitly recited.

The amount of organic acid or alkali metal salt thereof present in the aerosol generating material, relative to the substituted 3-(1-methylpyrrolidin-2-yl)pyridine, optionally substituted 3-(azetidin-2-yl)pyridine, optionally substituted 3-(azetidin-2-ylmethoxy)pyridine (e.g., 2-methyl-5-(1-methylpyrrolidin-2-yl)pyridine), or other active agent, may vary. Generally, as the concentration of the organic acid (or the conjugate base thereof) increases, the percent of substituted 3-(1-methylpyrrolidin-2-yl)pyridine, optionally substituted 3-(azetidin-2-yl)pyridine, optionally substituted 3-(azetidin-2-ylmethoxy)pyridine, or other active agent that is ion paired with the organic acid increases. This typically increases the partitioning of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent in the form of an ion pair, into octanol versus water as measured by the log P (the log₁₀ of the partitioning coefficient). In some embodiments, the aerosol generating material comprises from about 0.05, about 0.1, about 1, about 1.5, about 2, or about 5, to about 10, about 15, or about 20 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent, calculated as the free base of the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent.

In some embodiments, the aerosol generating material comprises from about 2 to about 10, or from about 2 to about 5 molar equivalents of the organic acid, the alkali metal salt thereof, or the combination thereof, relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent on a free-base basis. In some embodiments, the organic acid, the alkali metal salt thereof, or the combination thereof, is present in a molar ratio with the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent from about 2, about 3, about 4, or about 5, to about 6, about 7, about 8, about 9, or about 10. In embodiments wherein more than one organic acid, alkali metal salt thereof, or both, are present, it is to be understood that such molar ratios reflect the totality of the organic acids present. In some embodiments, the aerosol generating material comprises benzoic acid and sodium benzoate wherein a total amount of benzoate (i.e., benzoic acid and benzoate) is in a molar ratio in a range from about 3 to about 5 relative to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent. In some embodiments, the molar ratio of the total amount of benzoate to the substituted 3-(1-methylpyrrolidin-2-yl)pyridine, optionally substituted 3-(azetidin-2-yl)pyridine, optionally substituted 3-(azetidin-2-ylmethoxy)pyridine, or other active agent is about 3.2 or about 4.8.

In some embodiments, the organic acid inclusion is sufficient to provide a pH of from about 4.0 to about 9.0, such as from about 4.5 to about 7.0, or from about 5.5 to about 7.0, from about 4.0 to about 5.5, or from about 7.0 to about 9.0. Reference herein to “a pH” means the pH of an aqueous solution of the aerosol generating material prepared by dissolving or suspending 5 grams of aerosol generating material in 95 grams of water and measuring the pH of the resulting solution with a calibrated pH meter.

In some embodiments, the organic acid inclusion is sufficient to provide a pH of from about 4.5 to about 6.5, for example, from about 4.5, about 5.0, or about 5.5, to about 6.0, or about 6.5. In some embodiments, the desired pH is from about 4.5 to about 6.5, and the organic acid is provided in a quantity sufficient to provide such a pH. In some embodiments, the organic acid is provided in a quantity sufficient to provide a pH of from about 5.5 to about 6.5, for example, from about 5.5, about 5.6, about 5.7, about 5.8, about 5.9, or about 6.0, to about 6.1, about 6.2, about 6.3, about 6.4, or about 6.5.

In some embodiments, a mineral acid (e.g., hydrochloric acid, sulfuric acid, phosphoric acid, or the like), alone or in combination with an organic acid, is added to adjust the pH of the aerosol generating material to the desired value. In some embodiments, a buffer (e.g., a buffer as described herein below) is added to the aerosol generating material to the desired value, and/or to maintain the pH of the aerosol generating material at the desired value.

In some embodiments, the aerosol generating material further comprises a solubility enhancer to increase the solubility of one or more of the organic acid or salt thereof. Suitable solubility enhancers include, but are not limited to, humectants as described herein, such as glycerol or propylene glycol.

### Further Active Agents

In some embodiments, the aerosol generating material comprises an active agent in addition to the substituted 3-(1-methylpyrrolidin-2-yl)pyridine. The additional active agent may be a physiologically active material, which is a material intended to achieve or enhance a physiological response. The active agent may for example be selected from nutraceuticals, nootropics, psychoactives. The active agent may be naturally occurring or synthetically obtained. The active agent may comprise for example nicotine, caffeine, taurine, theanine, vitamins such as B6 or B12 or C, melatonin, cannabinoids, or constituents, derivatives, or combinations thereof. The active agent may comprise one or more constituents, derivatives or extracts of cannabis or another botanical material or extract thereof (other than tobacco).

### Nicotine Component

In some embodiments, the aerosol generating material comprises nicotine as an active agent. In some embodiments, the aerosol generating material has a nicotine content of from about 1.5 wt % to about 7 wt % (DWB).

In some embodiments, the aerosol generating material may comprise from at least about 1.5 wt %, about 2 wt %, about 2.5 wt %, about 3 wt %, about 3.5 wt %, about 4 wt %, about 4.5 wt % or about 5 wt % of nicotine (DWB). The aerosol generating material may comprise no more than about 7 wt %, about 6.5 wt %, about 6 wt %, about 5.5 wt %, about 5 wt %, about 4.5 wt %, about 4 wt %, about 3.5 wt % or about 3 wt % of nicotine (DWB). For example, the aerosol generating material may comprise from about 2 to about 6 wt %, or from about 4 to about 5 wt % nicotine by weight of the aerosol generating material (DWB).

In some embodiments, the aerosol generating material comprises from about 1 wt %, about 1.5 wt % or about 2 wt % to about 6 wt %, about 5 wt %, about 4 wt % or about 3 wt % of nicotine (DWB).

In some embodiments, the aerosol generating material of the disclosure can be completely free or substantially free of nicotine (3-(1-methylpyrrolidin-2-yl)pyridine). For example, some embodiments can have less than 0.01% by weight of nicotine, or less than 0.001% by weight of nicotine, or less than 0.0001%, or even 0% by weight of nicotine, calculated as the free base and based on the total weight of the aerosol generating material. In some embodiments the aerosol generating material is completely free of (R)-, (S)-, and (R S)-3-(1-methylpyrrolidin-2-yl)pyridine (e.g., having 0% by weight of nicotine, including racemic nicotine and nicotine enantiomers, calculated as the free base and based on the total weight of the aerosol generating material).

### Cannabinoids

In some embodiments, the active agent comprises one or more cannabinoids. As used herein, the term “cannabinoid” refers to a class of diverse natural or synthetic chemical compounds that acts on cannabinoid receptors (e.g., CB1 and CB2) in cells that alter neurotransmitter release in the brain. Cannabinoids are cyclic molecules exhibiting particular properties such as the ability to easily cross the blood-brain barrier. Cannabinoids may be naturally occurring (Phytocannabinoids) from plants such as cannabis, (endocannabinoids) from animals, or artificially manufactured (synthetic cannabinoids).

Cannabis species express at least 85 different phytocannabinoids, and these may be divided into subclasses, including cannabigerols, cannabichromenes, cannabidiols, tetrahydrocannabinols, cannabinols and cannabinodiols, and other cannabinoids, such as cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), and tetrahydrocannabivarinic acid (THCV A).

In some embodiments, the cannabinoid is selected from the group consisting of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabmolic acid (THCA), tetrahydrocannabivarinic acid (THCV A), and mixtures thereof.

In certain embodiments, the cannabinoid is selected from tetrahydrocannabinol (THC), the primary psychoactive compound in cannabis, and cannabidiol (CBD), another major constituent of the plant, but which is devoid of psychoactivity. All of the above compounds can be used in the form of an isolate from plant material or synthetically derived. Certain cannabinoids, including but not limited to CBD and THC, may exist in more than one isomeric form, for example Δ8- and Δ9-THC. Such isomeric forms may be naturally occurring or may be synthetic. For avoidance of doubt, reference within the present disclosure to a “cannabinoid” is intended to be inclusive of any and all isomeric forms thereof.

In some embodiments, the cannabinoid comprises at least tetrahydrocannabinol (THC). In some embodiments, the cannabinoid is tetrahydrocannabinol (THC). In some embodiments, the THC is Δ8-THC. In some embodiments, the THC is Δ9-THC.

In some embodiments, the cannabinoid comprises at least cannabidiol (CBD). In some embodiments, the cannabinoid is cannabidiol (CBD). In some embodiments, the CBD is synthetic CBD. In some embodiments, the CBD is Δ8-CBD. In some embodiments, the CBD is Δ9-CBD.

In some embodiments, the cannabinoid (e.g., CBD) is added to the aerosol generating material in the form of an isolate. An isolate is an extract from a plant, such as cannabis, where the active material of interest (in this case the cannabinoid, such as CBD) is present in a high degree of purity, for example greater than 95%, greater than 96%, greater than 97%, greater than 98%, or around 99% purity.

In some embodiments, the cannabinoid is an isolate of CBD in a high degree of purity, and the amount of any other cannabinoid in the aerosol generating material is no greater than about 1% by weight of the aerosol generating material, such as no greater than about 0.5% by weight of the aerosol generating material such as no greater than about 0.1% by weight of the aerosol generating aerosol generating material, such as no greater than about 0.01% by weight of the aerosol generating material.

The choice of cannabinoid and the particular percentages thereof which may be present within the aerosol generating material will vary depending upon the desired characteristics of the material.

In some embodiments, the cannabinoid (such as CBD) is present in the aerosol generating material in a concentration of at least about 0.001% by weight of the aerosol generating material, such as in a range from about 0.001% to about 2% by weight of the aerosol generating material. In some embodiments, the cannabinoid (such as CBD) is present in the aerosol generating material in a concentration of from about 0.1% to about 1.5% by weight, based on the total weight of the aerosol generating material. In some embodiments, the cannabinoid (such as CBD) is present in a concentration from about 0.4% to about 1.5% by weight, based on the total weight of the aerosol generating material.

Alternatively, or in addition to a cannabinoid, the active agent may include a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (tumeric), catechin, quercetin, salvinorin A, N-acylethanolamines, and N-alkylamide lipids. Such compounds can be used in the same amounts and ratios noted herein for cannabinoids.

### Terpenes

Active agents suitable for use in the aerosol generating material can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

In some embodiments, the terpene is a terpene derivable from a phytocannabinoid producing plant, such as a plant from the stain of the Cannabis sativa species, such as hemp. Suitable terpenes in this regard include so-called “C10” terpenes, which are those terpenes comprising 10 carbon atoms, and so-called “C15” terpenes, which are those terpenes comprising 15 carbon atoms. In some embodiments, the active agent comprises more than one terpene. For example, the active agent may comprise one, two, three, four, five, six, seven, eight, nine, ten or more terpenes as defined herein. In some embodiments, the terpene is selected from pinene (alpha and beta), geraniol, linalool, limonene, carvone, eucalyptol, menthone, iso-menthone, piperitone, myrcene, beta-bourbonene, germacrene and mixtures thereof.

Terpenes and/or cannabinoids may be present as an active agent, as an aerosol former, or as a flavorant. The amounts of terpenes and/or cannabinoids present may vary accordingly based on their intended purpose.

### Flavoring Agent

In some embodiments, the aerosol generating material as described herein comprises a flavoring agent. As used herein, a “flavoring agent” or “flavorant” is any flavorful or aromatic substance capable of altering the sensory characteristics associated with the aerosol generating material or the vapor produced therefrom. Examples of sensory characteristics that can be modified by the flavoring agent include taste, mouthfeel, moistness, coolness/heat, and/or fragrance/aroma. Flavoring agents may be natural or synthetic, and the character of the flavors imparted thereby may be described, without limitation, as fresh, sweet, herbal, confectionary, floral, fruity, or spicy.

Flavoring agents may be imitation, synthetic or natural ingredients or blends thereof. Flavoring agents may include naturally occurring flavor materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents.

In some embodiments, the flavor comprises menthol, spearmint and/or peppermint. In some embodiments, the flavor comprises flavor components of cucumber, blueberry, citrus fruits and/or redberry. In some embodiments, the flavor comprises eugenol. In some embodiments, the flavor comprises flavor components extracted from tobacco. In some embodiments, the flavor comprises flavor components extracted from cannabis, such as terpenes. In some embodiments, the flavor comprises menthol.

Flavorants may further include flavor enhancers, sensorial receptor site activators or stimulators, and trigeminal sensates, As used herein, “trigeminal sensate” refers to a flavoring agent which has an effect on the trigeminal nerve, producing sensations including heating, cooling, tingling, and the like. Non-limiting examples of trigeminal sensate flavoring agents include capsaicin, citric acid, menthol, Sichuan buttons, erythritol, and cubebol.

In some embodiments, the aerosol generating material comprises a sensate which provides to the user of such aerosol generating material a cooling effect. Suitable cooling agents include, but are not limited to, menthane, menthone, menthone ketals, menthone glycerol ketals, substituted p-menthanes, acyclic carboxamides, monomenthyl glutarate, substituted cyclohexanamides, substituted cyclohexane carboxamides, substituted ureas and sulfonamides, substituted menthanols, hydroxymethyl and hydroxymethyl derivatives of p-menthane, 2-mercapto-cyclo-decanone, hydroxycarboxylic acids with 2-6 carbon atoms, cyclohexanamides, menthyl acetate, menthyl salicylate, N-ethyl-p-menthane-3-carboxamide (WS-3), ethyl ester of N-[[5-methyl-2-(1-methylethyl)cyclohexyl]carbonyl]glycine (WS-5), WS-14, N,2,3-trimethyl-2-isopropyl butanamide (WS-23), WS-27, WS-30, (−)-Menthyloxyethanol (Coolact® 5), WS-NA (FEMA 4693), WS-116 (FEMA 4603), N-ethyl-2,2-diisopropylbutanamide, isopulegol, menthyloxy propane diol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, menthyl glutarate, N,2,3-trimethyl-2-(1-methylethyl)-butanamide, N-ethyl-trans-2-cis-6-nonadienamide, N,N-dimethyl menthyl succinamide, N-(2-hydroxyethyl)-2,3-dimethyl-2-isopropylbutanamide, substituted p-menthanes, substituted p-menthane-carboxamides, 2-isopropanyl-5-methylcyclohexanol, menthyl ethylene glycol carbonate, menthone glycerol ketals (e.g., menthone 1,2-glycerol ketal), menthone (S)-lactic acid ketal, menthyl acetoacetate, 3-1-menthoxypropane-1,2-diol, menthyl lactate, eucalyptus extract, menthol propylene glycol carbonate, menthol ethylene glycol carbonate, menthol glyceryl ether, N-tert-butyl-p-menthane-3-carboxamide, p-menthane-3-carboxylic acid glycerol ester, methyl-2-isopropyl-bicyclo[2.2.1]heptane-2-carboxamide, (1R,2S,5R)—N-(4-(carbamoylmethyl)phenyl)-menthylcarboxamide, 2-[2-(p-menthan-3-yloxy)ethoxy]ethanol, (1R,2R,4R)-1-(2-Hydroxy-4-methylcyclohexyl)ethenone, 2-(p-tolyloxy)-N-(1H-pyrazol-5-yl)-N-((thiophen-2-yl)methyl)acetamide, menthol methyl ether, menthyl pyrrolidone carboxylate, 2,5-dimethyl-4-(1-pyrrolidinyl)-3(2H)-furanone, cyclic α-keto enamines, and cyclotene derivatives (e.g., 3-methyl-2-(1-pyrrolidinyl)-2-cyclopenten-1-one and 5-methyl-2-(1-pyrrolidinyl)-2-cyclopenten-1-one). Other compounds include the alpha-keto enamines disclosed in U.S. Pat. No. 6,592,884 to Hofmann et al., which is incorporated in its entirety herein. These and other suitable cooling agents are further described in the following U.S. patents, all of which are incorporated in their entirety by reference hereto: U.S. Pat. Nos. 4,230,688; 4,032,661; 4,459,425; 4,178,459; 4,296,255; 4,136,163; 5,009,893; 5,266,592; 5,698,181; 6,277,385; 6,627,233; 7,030,273. Still other suitable cooling agents are further described in US Patent Application Publications Nos. 2005/0222256 and 2005/0265930, each of which are incorporated in their entirety by reference hereto. In some embodiments, the cooling agent comprises menthol, eucalyptus, mint, menthol, menthyl esters, eucolyptol, WS-3, WS-23, WS-5, (1R,2S,5R)—N-(4-(cyanomethyl)phenyl)menthylcarboxamide (Evercool™ 180), (1R,2S,5R)—N-(2-(pyridin-2-yl)ethyl)menthylcarboxamide (Evercool™ 190), or a combination thereof.

In some embodiments, the aerosol generating material does not comprise a flavoring agent, and comprises only a cooling agent to provide the desired user experience. In some embodiments, the cooling agent is WS-3.

In some embodiments, the aerosol generating material comprises a modulator or sensate which provides to the user of such aerosol generating material a warming effect. Suitable warming agents include, but are not limited to, ethers of vanillyl alcohol (e.g., methyl, ethyl, n-propyl, isopropyl, n-butyl, isobutyl, isoamyl, n-hexyl), gingerol, shogaol, paradol, zingerone, capsaicin, dihydrocapsaicin, nordihydrocapsaicin, homocapsaicin, homodihydrocapsaicin, benzyl alcohol, and combinations thereof. In some embodiments, the warming agent comprises vanillyl butyl ether, vanillyl ethyl ether, capsaicin, or a combination thereof.

Flavoring agents may be in any suitable form, for example, a liquid such as an oil, or a solid such as a powder or wax. In some instances, the flavoring agent may be provided in a spray-dried form or a liquid form.

The amount of flavoring agent utilized in the aerosol generating material can vary, but is typically up to about 60% by weight. For example, aerosol generating material may comprise up to about 60 wt %, about 50 wt %, about 40 wt %, about 30 wt %, about 20 wt %, about 10 wt % or about 5 wt % of a flavoring agent. In some embodiments, the aerosol generating material may comprise at least about 0.5 wt %, about 1 wt %, about 2 wt %, about 5 wt %, about 10 wt %, about 20 wt % or about 30 wt % of flavoring agent (calculated on a dry weight basis). For example, the aerosol generating material may comprise from about 10 to about 60 wt %, from about 20 to about 50 wt % or from about 30 to about 40 wt % of flavoring agent.

In some embodiments, the flavor comprises flavor components of berry fruits, citrus fruits, and/or tropical fruits. In some embodiments, the flavor comprises cucumber, blueberry, citrus fruits, pineapple, strawberry, and/or redberry. In some embodiments, the flavor comprises flavor components of cucumber, blueberry, citrus fruits and/or redberry. In some embodiments, the flavor comprises eugenol.

In some embodiments, the flavor comprises menthol, spearmint and/or peppermint. In some embodiments, the flavoring agent (if present) comprises, consists essentially of, or consists of, menthol. In some embodiments, the aerosol generating material does not comprise an added flavor.

### Water Content

In some embodiments, the aerosol generating material has a water content of between about 3% to about 20% by weight. For example, the aerosol generating material has a water content of between about 5% to about 15% by weight, for example between about 8% and about 14% by weight. In some embodiments, the aerosol generating material comprises a water content of about 10-12% by weight.

### Form of Aerosol Generating Material

The aerosol generating material of the disclosure may, for example, be in the form of a solid, liquid or semi-solid (such as a gel). In some embodiments, the aerosol generating material is in the form of a sheet, such as a cast or extruded sheet. The sheet may be in the form of a wrapper, it may be gathered to form a gathered sheet or it may be shredded to form a shredded sheet. The shredded sheet may comprise one or more strands or strips of aerosol generating material.

In some embodiments, the aerosol generating material (e.g., in sheet form) has a thickness from about 100 μm to about 700 μm, such as from about 150 μm to about 450 μm. In some embodiments, the aerosol generating material has a thickness from about 150 μm to about 300 μm.

In some embodiments, the aerosol generating material has a filling value from about 2 cm³/g to about 10 cm³/g. In some embodiments, the aerosol generating material has a filling value of from about 3 cm³/g to about 8 cm³/g, for example the aerosol generating material may have a filling value of from about 4 cm³/g to about 7 cm³/g, such as from about 4 cm³/g to about 6 cm³/g. In some embodiments, the aerosol generating material has a filling value of about 5 cm³/g. When the filling value is between about 2 cm³/g to about 10 cm³/g, for example about 5 cm³/g, a consumable containing the aerosol generating material can weigh less. Therefore, the overall weight of material used, to achieve the same/required filling value, is less, which can provide a saving to the cost of goods. In addition, using the aerosol generating material described herein may also be advantageous because the resistance to draw is not affected, therefore providing the consumer with a favorable user experience.

In some embodiments, the aerosol generating material exhibits a bursting strength of between about 5 KPa and about 40 KPa. The burst strength can be measured using known methods. A suitable method is described in, for example, US Patent Application Publication No. 2024/041090, incorporated by reference herein with respect to determination of burst strength. For example, burst strength may be measured using a calibrated Texture Analyser (50 kg load cell, 20 mm probe height calibration, 1 g contact force) and Exponent software (Stable Micro Systems) using a 3 cm² sheet of aerosol generating material and a 5 mm stainless steel ball probe. In some embodiments, the aerosol generating material exhibits a bursting strength of between about 10 KPa and about 30 KPa. In some embodiments, the aerosol generating material exhibits a bursting strength of between about 15 Pa and about 25 KPa, for example between about 20 KPa and about 25 KPa. In some embodiments, the bursting strength is about 20 KPa.

In some embodiments, the sheet or shredded sheet comprises a first surface and a second surface opposite the first surface. The dimensions of the first and second surfaces are congruent. The first and second surfaces of the sheet or shredded sheet may have any shape. For example, the first and second surfaces may be square, rectangular, oblong or circular. Irregular shapes are also envisaged.

The first and/or second surfaces of the sheet or shredded sheet may be relatively uniform (e.g., they may be relatively smooth) or they may be uneven or irregular. For example, the first and/or second surfaces of the sheet may be textured or patterned to define a relatively coarse surface. In some embodiments, the first and/or second surfaces are relatively rough.

The smoothness of the first and second surfaces may be influenced by a number of factors, such as the area density of the sheet or shredded sheet, the nature of the components that make up the aerosol generating material or whether the surfaces of the material have been manipulated, for example embossed, scored or otherwise altered to confer them with a pattern or texture.

The areas of the first and second surfaces are each defined by a first dimension (e.g., a width) and a second dimension (e.g., a length). The measurements of the first and second dimensions may have a ratio of 1:1 or greater than 1:1 and thus the sheet or shredded sheet may have an “aspect ratio” of 1:1 or greater than 1:1. As used herein, the term “aspect ratio” is the ratio of a measurement of a first dimension of the first or second surface to a measurement of a second dimension of the first or second surface. An “aspect ratio of 1:1” means that a measurement of the first dimension (e.g., width) and a measurement of the second dimension (e.g., length) are identical. An “aspect ratio of greater than 1:1” a measurement of the first dimension (e.g., width) and a measurement of the second dimension (e.g., length) are different. In some embodiments, the first and second surfaces of the sheet or shredded sheet have an aspect ratio of greater than 1:1, such as 1:2, 1:3, 1:4, 1:5, 1:6, 1:7 or more.

The shredded sheet may comprise one or more strands or strips of the aerosol generating material. In some embodiments, the shredded sheet comprises a plurality (e.g., two or more) strands or strips of the aerosol generating material. The strands or strips of aerosol generating material may have an aspect ratio of 1:1. In an embodiment, the strands or strips of aerosol generating material have an aspect ratio of greater than 1:1. In some embodiments, the strands or strips of aerosol generating material have an aspect ratio of from about 1:5 to about 1:16, or about 1:5, 1:6, 1:7, 1:8, 1:9, 1:10, 1:11 or 1:12. Where the aspect ratio of the strands or strips is greater than 1:1, the strands or strips comprises a longitudinal dimension, or length, extending between a first end of the strand or strip and a second end of the strand or strip.

Where the shredded sheet comprises a plurality of strands or strips of material, the dimensions of each strand or strip may vary between different strands or strips. For example, the shredded sheet may comprise a first population of strands or strips and a second population of strands or strips, wherein the dimensions of the strands or strips of the first population are different to the dimensions of the strands or strips of the second population. In other words, the plurality of strands or strips may comprise a first population of strands or strips having a first aspect ratio and a second population of strands or strips having a second aspect ratio that is different to the first aspect ratio.

A first dimension, or cut width, of the strands or strips of aerosol generating material is between 0.9 mm and 1.5 mm. The inventors have found that, when strands or strips of aerosol generating material having a cut width of below 0.9 mm are incorporated into an article for use in a non-combustible aerosol provision system, the pressure drop across the article may be increased to a level that renders the article unsuitable for use in a non-combustible aerosol-provision device. However, if the strands or strips have a cut width above 2 mm (e.g., greater than 2 mm), then it may be challenging to insert the strands or strips of aerosol generating material into the article during its manufacture. In a preferred embodiment, the cut width of the strands or strips of aerosol generating material is between about 1 mm and 1.5 mm.

The strands or strips of material are formed by shredding the sheet of aerosol generating material. The sheet of aerosol generating material may be cut widthwise, for example in a cross-cut type shredding process, to define a cut length for the strands or strips of aerosol generating material, in addition to a cut width. The cut length of the shredded aerosol generating material is preferably at least 5 mm, for instance at least 10 mm, or at least 20 mm. The cut length of the shredded aerosol generating material can be less than 60 mm, less than 50 mm, or less than 40 mm.

In some embodiments, a plurality of strands or strips of aerosol generating material is provided and at least one of the plurality of strands or strips of aerosol generating material has a length greater than about 10 mm. At least one of the plurality of strands or strips of aerosol generating material can alternatively or in addition have a length between about 10 mm and about 60 mm, or between about 20 mm and about 50 mm. Each of the plurality of strands or strips of aerosol generating material can have a length between about 10 mm and about 60 mm, or between about 20 mm and about 50 mm.

### Preparation of Aerosol Generating Material

As described herein, the aerosol generating material is typically in the form of a sheet, such as a cast or extruded sheet. The aerosol generating material in sheet form may be prepared according to typical band casting, extrusion, or paper recon methods.

In some embodiments, the aerosol generating material is prepared by a band casting method. The band casting method of preparing an aerosol generating material of the disclosure generally comprises the steps of combining a fibrous plant material as described herein, a substituted 3-(1-methylpyrrolidin-2-yl)pyridine as described herein, a binder as described herein, water, and optionally an aerosol former as described herein to form a slurry; casting the slurry onto a surface to form a sheet of the aerosol generating material; and drying the sheet of aerosol generating material. A suitable band casting method is described in, for example, International Patent Application Publication No. WO2024/161119, incorporated by reference herein with respect to band casting methodology. As an alternative to or in addition to adding the substituted 3-(1-methylpyrrolidin-2-yl)pyridine to the slurry components, at least a portion of the substituted 3-(1-methylpyrrolidin-2-yl)pyridine may be applied during or after drying (e.g., by spraying it on or wiping it onto the sheet). The substituted 3-(1-methylpyrrolidin-2-yl)pyridine is generally applied as a solution in, for example, an aerosol former such as glycerol, propylene glycol, or a mixture thereof. Without wishing to be bound by theory, it is believed that adding the substituted 3-(1-methylpyrrolidin-2-yl)pyridine solely after sheet formation, either during or after drying, may reduce the potential for contamination and/or increase the homogeneity of distribution of the substituted 3-(1-methylpyrrolidin-2-yl)pyridine in the aerosol generating material.

In some embodiments, the aerosol generating material is prepared by a paper recon method. The paper recon method of preparing an aerosol generating material of the disclosure generally comprises the steps of mixing a portion of fibrous plant material as described herein with a solvent such as water in order to extract the soluble components contained therein; separating the soluble extract from the fibrous portion by physical separation; mixing and refining the fibrous portion to produce a refined pulp, for example by chemically treating the fibrous portion; passing the refined pulp into a papermaking machine in order to form a base sheet; impregnating the base sheet with a substituted 3-(1-methylpyrrolidin-2-yl)pyridine and aerosol former, each as described herein; and drying the impregnated sheet to form the aerosol generating material. A suitable paper recon method is described in, for example, International Patent Application Publication No. WO2024/149846, incorporated by reference herein with respect to paper recon methodology. As described with respect to the band casting method, at least a portion of the substituted 3-(1-methylpyrrolidin-2-yl)pyridine may be applied during or after drying of the sheet as an alternative or in addition to incorporating into the base sheet before drying and provides the same potential advantages.

In some embodiments, the aerosol generating material is prepared by an extrusion method, e.g., as an extruded sheet or extruded granule form). The extrusion method of preparing an aerosol generating material of the disclosure generally comprises the steps of preparing a mixture comprising the fibrous plant material, binder, substituted 3-(1-methylpyrrolidin-2-yl)pyridine, and aerosol former, each as described herein; extruding the mixture through a die; and drying the extruded mixture. A suitable extrusion method is described in, for example, International Patent Application Publication Nos. WO2020/14853 and WO2023/118840, each of which is incorporated by reference herein with respect to extrusion methodology. As described with respect to the band casting and paper recon methods, at least a portion of the substituted 3-(1-methylpyrrolidin-2-yl)pyridine may be applied during or after drying of the sheet as an alternative or in addition to incorporating into the mixture before extrusion and provides the same potential advantages.

In some embodiments, regardless of which above-noted process is used to form the aerosol generating material in the form of a sheet, the aerosol generating material is shredded into strands or strips of aerosol generating material.

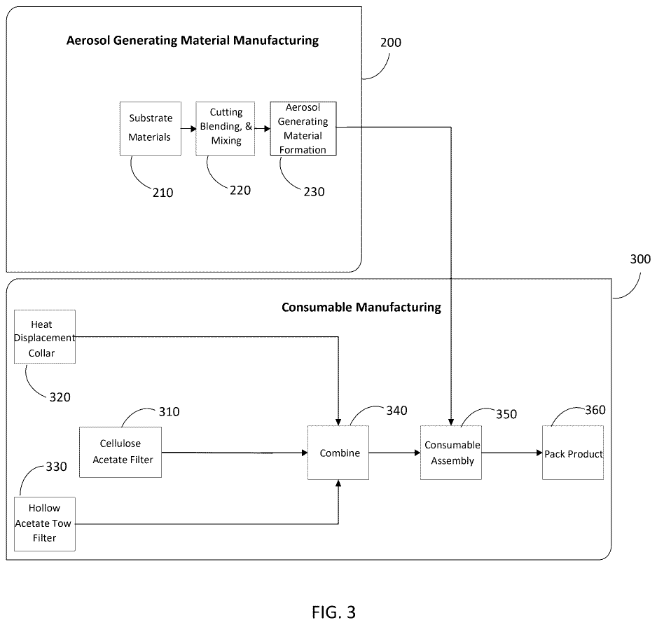

In some embodiments, the aerosol generating material is formed using the process illustrated in block 200 of FIG. 3. As shown, the process can include providing substrate materials in operation 210, which can be, for example, any of the fibrous plant materials disclosed herein such as tobacco materials, non-tobacco botanical materials (e.g., rooibos), or combinations thereof. The substrate materials can also include fillers as described herein, such as wood pulp or wood fibers. The substrate materials are cut and blended and mixed with other components of the aerosol generating material (e.g., aerosol formers, binders, or flavorants) in operation 220 and then formed into an aerosol generating material at operation 230. As discussed previously, the formation step can include, for example, forming a sheet or extrudate material and optionally shredding of the material to form a shredded aerosol generating material.

The substituted 3-(1-methylpyrrolidin-2-yl)pyridine (or other active agents) can be added at any step noted in block 200 of FIG. 3. For example, the substituted 3-(1-methylpyrrolidin-2-yl)pyridine could be sprayed on individual substrate materials prior to the cutting, blending, and mixing at operation 220, or at any stage during the cutting, blending, or mixing steps. In some embodiments, the substituted 3-(1-methylpyrrolidin-2-yl)pyridine can be added after the cutting, blending, and mixing at operation 220, such as during aerosol generating material formation at operation 230. For example, the substituted 3-(1-methylpyrrolidin-2-yl)pyridine can be added to a slurry formed prior to sheet or extrudate formation, sprayed on a sheet material, or sprayed on a shredded material formed from a sheet or extruded material during operation 230.

### Consumables and Articles

In another aspect is provided an aerosol generating rod comprising the aerosol generating material as disclosed herein. As used herein, the term “rod” is used to describe a generally cylindrical element of substantially circular, oval, or elliptical cross section. The aerosol generating rod may have a total weight of between about 250 mg and about 350 mg. The aerosol generating rod may have an outer circumference of at least about 19 mm, preferably between about 19 mm and about 23 mm or about 21 mm. This may facilitate insertion of the article into an aerosol generation device.

In another aspect is provided an article comprising the aerosol generating material (e.g., an aerosol generating rod) as disclosed herein. An article may also be referred to as a “consumable”. As used herein, a “consumable” is an article comprising or consisting of aerosol generating material, part or all of which is intended to be consumed during use by a user. A consumable, such as a consumable for use with a non-combustible aerosol provision device, may comprise one or more other components, such as, an aerosol generating material storage area, an aerosol generating material transfer component, an aerosol generator, an aerosol generation area, a housing, a wrapper, a filter, and/or a mouthpiece. A consumable may also comprise an aerosol generator, such as a heater, that emits heat to cause the aerosol generating material to generate aerosol in use. The heater may, for example, comprise combustible material, a material heatable by electrical conduction, or a susceptor. The consumable may be any shape or size that is appropriate to the smoking device. In some embodiments, the consumable is a rod shape.

In some embodiments, the aerosol generating material may be positioned in an aerosol generating section of an article. When in use, the aerosol generating section may exhibit a pressure drop of from about 15 to about 40 mm H₂O. In some embodiments, the aerosol generating section exhibits a pressure drop across the aerosol generating section of from about 15 to about 30 mm H₂O.

In some embodiments, the article comprises a mouthpiece, and a cylindrical rod of aerosol generating material as disclosed herein connected, either directly or indirectly, to the mouthpiece. In some embodiments, the mouthpiece is positioned downstream of the aerosol generating material.

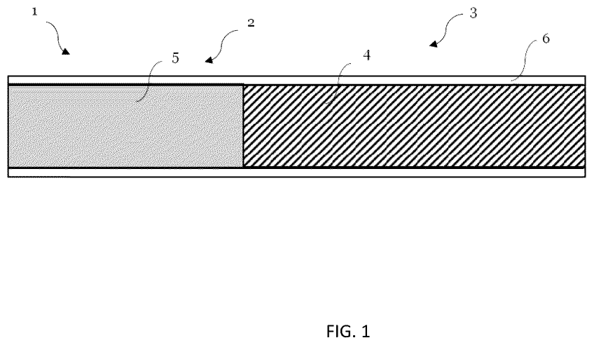

FIG. 1 is a side-on cross-sectional view of a consumable or article 1 for use in an aerosol delivery system according to a non-limiting embodiment of the disclosure. With reference to FIG. 1, The article 1 comprises a mouthpiece segment 2, and an aerosol-generating segment 3. The aerosol generating segment 3 is in the form of a cylindrical rod and comprises an aerosol generating material 4. The aerosol generating material can be any of the materials discussed herein. Although described above in rod form, the aerosol generating segment 3 can be provided in other forms, for instance a plug, pouch, or packet of material within an article. The mouthpiece segment 2, in the illustrated embodiment, includes a body of material 5 such as a fibrous or filamentary tow.

In the illustrated embodiment, the rod-shaped consumable 1 further comprises a wrapper 6 circumscribing the mouthpiece segment 2 and aerosol generating segment 3, such as a paper wrapper. In some embodiments, wrapper has a permeability of less than 100 Coresta Units.

The mouthpiece wrapper, also described herein as a tipping paper, may be wrapped around the full length of the mouthpiece segment 2 and over part of the rod of aerosol generating material 4 and has an adhesive on its inner surface to connect the mouthpiece segment 2 and rod 3. In some embodiments, the wrapper 6 comprises a sensate such as a flavorant, an active agent, or both. The wrapper 6 may comprise an inwardly facing surface and an outwardly facing surface and the sensate may be present on at least a portion of the inwardly facing surface and/or the outwardly facing surface of the wrapper 6. For example, the sensate may be disposed on an outwardly facing surface of the mouthpiece wrapper in an area which comes into contact with the consumer's lips during use. By disposing the sensate on the outwardly facing surface of the mouthpiece wrapper, the sensate may be transferred to the consumer's lips during use. Transfer of the sensate to the consumer's lips during use of the article may modify the organoleptic properties (e.g., taste) of the aerosol generated by the aerosol generating material. For example, the sensate may impart flavor to the aerosol generated by the aerosol generating material 4. The sensate may be at least partially soluble in water such that it may be transferred to the user via the consumer's saliva.

In some embodiments, the sensate comprises a flavorant as described herein. In some embodiments, the flavorant comprises licorice, rose oil, vanilla, lemon oil, orange oil, a mint flavor, suitably menthol and/or a mint oil from any species of the genus Mentha such as peppermint oil and/or spearmint oil, or lavender, fennel, or anise. In some embodiments, the sensate comprises sugars and/or sugar substitutes (e.g., sucralose, acesulfame potassium, aspartame, saccharine, cyclamates, lactose, sucrose, glucose, fructose, sorbitol, or mannitol). Additionally or alternatively, the sensate may comprise a material that delivers a cooling, heating or sour sensation to the consumer during use of the article.

The mouthpiece wrapper 6 may be arranged to wrap around and enclose the mouthpiece 2 in a region between the upstream end and downstream end of the mouthpiece. The wrapper 6 may be arranged such that, when the article 1 is inserted into a heating device, a portion of the wrapper 6 is heated to the same or a similar temperature as the aerosol generating material 3.

In some embodiments, the wrapper (e.g., tipping paper) 6 extends 5 mm over the rod of aerosol generating material 3, but it can alternatively extend between 3 mm and 10 mm over the rod 3, or more preferably between 4 mm and 6 mm, to provide a secure attachment between the mouthpiece and rod. The wrapper (e.g., tipping paper) 6 can have a basis weight of, for instance, 40 gsm to 80 gsm, or between 50 gsm and 70 gsm, such as 58 gsm. These ranges of basis weights have been found to result in tipping papers having acceptable tensile strength while being flexible enough to wrap around the article 1 and adhere to itself along a longitudinal lap seam on the paper. The outer circumference of the wrapper (e.g., tipping paper) 6, once wrapped around the mouthpiece 2, may be about 21 mm.

### Preparation of Consumables

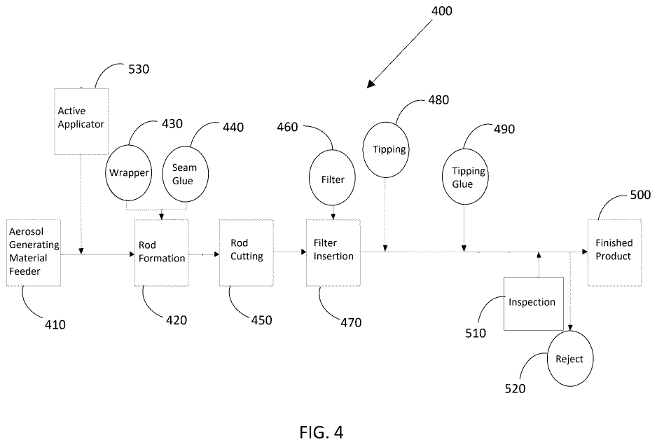

An example process for forming a consumable is shown in block 300 of FIG. 3. The non-limiting illustrated process utilizes a three-segment filter element that includes a central cellulose acetate tow section 310, an upstream heat displacement collar section (e.g., a hollow tube) 320, and a mouth-end hollow cellulose acetate tow section 330, which are combined to form a three-segment filter element at operation 340. Other filter element configurations could be used without departing from the present disclosure. Thereafter, the filter element is combined with the aerosol generating material and other components at operation 350, which is described in greater detail in FIG. 4. Thereafter, the finished product can be packaged at operation 360.

FIG. 4 illustrates an example method 400 for combining the consumable components into a finished product. As shown, the aerosol generating material (e.g., as formed in FIG. 3) is fed using feeder 410 (e.g., a conveyer belt) to a rod formation operation 420 where the aerosol generating material is enwrapped with a wrapper 430 that is sealed using seam glue 440. The formed rod is cut into desired lengths at operation 450 and each rod portion is combined with a filter element (e.g., as formed in FIG. 3) 460 at operation 470. Thereafter, a tipping material 480 is added to attach the filter element to the aerosol generating material rod and sealed using tipping glue 490 to form the finished product 500. In some embodiments, the process further comprises an inspection operation 510, such as inspecting the formed consumable to confirm proper rod construction and wrapper sealing. Any rejects 520 resulting from the inspection can be discarded. In some embodiments, the substituted 3-(1-methylpyrrolidin-2-yl)pyridine (or other active agents) can be added using an applicator 530 immediately upstream of rod formation 420. In this manner, the substituted 3-(1-methylpyrrolidin-2-yl)pyridine can be added to the aerosol generating material as the aerosol generating material is conveyed to the rod formation operation 420. An example applicator 530 is the Flavor Application System (FAS) available from Koehl Maschinenbau AG of Luxembourg, which provides metered dosing of the active agent and is configured to automatically adjust to manufacturing line speeds to maintain the desired dose per rod. Applying the substituted 3-(1-methylpyrrolidin-2-yl)pyridine immediately upstream of the rod formation 420 can be advantageous to avoid or minimize loss of the active agent during manufacturing, as may be seen with addition of the active agent during upstream manufacturing process steps as described above. Use of metered dosing of the active agent is advantageous for precise dosing of the active agent in each rod. In some embodiments, the application rate of the applicator 530 can be set over a wide range, such as 1 g/min to 480 g/min. As used herein, “metered dosing” refers to adding the active agent at a desired dose per consumable with a high degree of precision. For example, in some embodiments, the process of the disclosure provides metered dosing of the substituted 3-(1-methylpyrrolidin-2-yl)pyridine in a range from about 0.1 to about 5 mg, or from about 0.3 to about 2 mg per consumable rod. In some embodiments, the metered dose is about 0.5 mg to about 1.5 mg per consumable rod. In some embodiments, the precision of the metered dosing is within +/−10% of the desired amount, meaning the variance in dosing for each consumable rod is no more than +/−10%.

### Aerosol Delivery Systems

In another aspect is provided an aerosol delivery system comprising the aerosol generating material as disclosed herein, such as in the form of an aerosol generating rod. As used herein, the term “delivery system” is intended to encompass systems that deliver a substance to a user, and includes:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material); and
non-combustible aerosol provision systems that release compounds from an aerosol generating material without combusting the aerosol-generating material, such as electronic cigarettes, tobacco heating products, and hybrid systems to generate aerosol using a combination of aerosol-generating materials.

As used herein, a “combustible” aerosol provision system is one where a constituent aerosol generating material of the aerosol provision system (or component thereof) is combusted or burned during use in order to facilitate delivery of at least one substance to a user. In some embodiments, the delivery system is a combustible aerosol provision system, such as a system selected from the group consisting of a cigarette, a cigarillo and a cigar. Combustible delivery systems may further include additional components such as a filter, a filter rod, a filter segment, a tobacco rod, a spill, or a paper such as a plug wrap, a tipping paper or a cigarette paper. In some embodiments, it is envisaged that consumables which themselves comprise a means for powering an aerosol generating component may themselves form the non-combustible aerosol provision system.

In some embodiments, the delivery system is a non-combustible aerosol provision system, such as a powered non-combustible aerosol provision system. According to the present disclosure, a “non-combustible” aerosol provision system is one where a constituent aerosol generating material of the aerosol provision system (or component thereof) is not combusted or burned in order to facilitate delivery to a user. Typically, the non-combustible aerosol provision system comprises a non-combustible aerosol provision device, also referred to herein as an aerosol generation device, and a consumable as disclosed herein.

In some embodiments, the non-combustible aerosol provision system is an electronic cigarette, also known as a vaping device or electronic nicotine delivery system (END) although it is noted that the presence of nicotine in the aerosol-generating material is not a requirement.

In some embodiments, the non-combustible aerosol provision system is an aerosol generating material heating system, also known as a heat-not-burn system. An example of such a system is a tobacco heating system.

In some embodiments, the non-combustible aerosol provision system is a hybrid system to generate aerosol using a combination of aerosol generating materials, one or a plurality of which may be heated. Each of the aerosol generating materials may be, for example, in the form of a solid, liquid or gel. In some embodiments, the hybrid system comprises a liquid or gel aerosol generating material and a solid aerosol generating material as disclosed herein.

In some embodiments, the non-combustible aerosol provision system comprises an area for receiving the consumable, an aerosol generator, an aerosol generation area, a housing, a mouthpiece, and/or a filter.

An aerosol generator is a heater capable of interacting with the aerosol generating material so as to release one or more volatiles from the aerosol generating material to form an aerosol. In some embodiments, the aerosol generator is capable of generating an aerosol from the aerosol generating material without heating. For example, the aerosol generator may be capable of generating an aerosol from the aerosol generating material without applying heat thereto, for example via one or more of vibrational, mechanical, pressurization or electrostatic means.

In some embodiments, the system comprises an article as described herein and the aerosol generation device is arranged to receive at least a portion of the article comprising the aerosol generating material and to heat the portion of the article comprising the aerosol generating material and generate an aerosol from the aerosol generating material. The heater may, for example, comprise combustible material, a material heatable by electrical conduction, or a susceptor. A susceptor is a material that is heatable by penetration with a varying magnetic field, such as an alternating magnetic field. The susceptor may be an electrically conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically conductive and magnetic, so that the susceptor is heatable by both heating mechanisms. The device that is configured to generate the varying magnetic field is referred to herein as a magnetic field generator.

In some embodiments, the non-combustible aerosol provision system comprises a power source and a controller. The power source may be an electric power source or an exothermic power source. In some embodiments, the exothermic power source comprises a carbon substrate which may be energized so as to distribute power in the form of heat to the aerosol generating material or heat transfer material in proximity to the exothermic power source. In some embodiments, the power source, such as an exothermic power source, is provided in the article so as to form the non-combustible aerosol provision system.

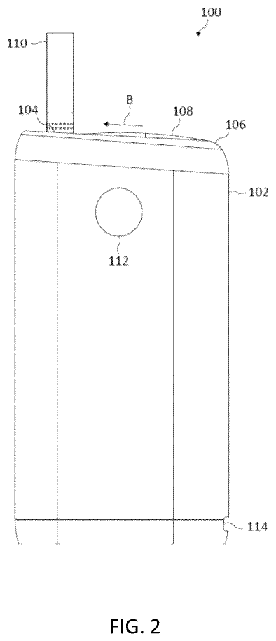

FIG. 2 shows an example of a non-combustible aerosol provision device 100 for generating aerosol from an aerosol generating material, such as the aerosol generating material of an article (i.e., a consumable) 110, each as described herein. In broad outline, the device 100 may be used to heat a replaceable article 110 comprising the aerosol generating material, for instance an article 1 as illustrated in FIG. 1 or as described elsewhere herein, to generate an aerosol or other inhalable medium which is inhaled by a user of the device 100. The device 100 and replaceable article 110 together form a system.

The device 100 comprises a housing 102 (in the form of an outer cover) which surrounds and houses various components of the device. The device 100 has an opening 104 in one end, through which the article 110 may be inserted for heating by a heating assembly. In use, the article 110 may be fully or partially inserted into the heating assembly where it may be heated by one or more components of the heater assembly.

The device 100 of this example comprises a first end member 106 which comprises a lid 108 which is moveable relative to the first end member 106 to close the opening 104 when no article 110 is in place. In FIG. 2, the lid 108 is shown in an open configuration, however the lid 108 may move into a closed configuration. For example, a user may cause the lid 108 to slide in the direction of arrow “B”.

The device 100 may also include a user-operable control element 112, such as a button or switch, which operates the device 100 when pressed. For example, a user may turn on the device 100 by operating the switch 112.

The device 100 may also comprise an electrical component, such as a socket/port 114, which can receive a cable to charge a battery of the device 100. For example, the socket 114 may be a charging port, such as a USB charging port.

Many modifications and other implementations of the disclosure will come to mind to one skilled in the art to which this disclosure pertains having the benefit of the teachings presented in the foregoing descriptions and the associated figures. Therefore, it is to be understood that the disclosure is not to be limited to the specific implementations disclosed herein and that modifications and other implementations are intended to be included within the scope of the appended claims. Although specific terms are employed herein, they are used in a generic and descriptive sense only and not for purposes of limitation.

### EXAMPLE

### Example 1. Preparation of an Aerosol Generating Material

An aerosol generating material according to a non-limiting embodiment of the disclosure is prepared. The aerosol generating material has the composition illustrated in Table 2.

TABLE 2
Aerosol generating material composition

| | % by weight of aerosol |
|---|---|
| Component | generating material |
| Fibrous plant material (e.g., rooibos) | 48-72 |
| Wood pulp/wood fiber | 10-14 |
| Botanical extract (e.g., rooibos) | 8-12 |
| Aerosol former (e.g., glycerol, propylene | 12-18 |
| glycol, or mixture thereof) | |
| Substituted 3-(1-methylpyrrolidin-2- | 0.01-5 |
| yl)pyridine | |
| Organic acid | 0-5 |
| Flavorant | 0-5 |

The aerosol generating material is prepared by adding a plant material (e.g., rooibos) to an extraction solvent and the mixture separated into a fibrous portion and an extract. The fibrous portion is mixed with wood pulp and refined to produce a refined pulp and formed into a sheet. The sheet is impregnated with the substituted 3-(1-methylpyrrolidin-2-yl)pyridine, aerosol former, extract, and optionally organic acid and flavorant and the mixture processed to form an impregnated sheet. The impregnated sheet is dried to form the aerosol generating material.

1. An aerosol generating material comprising a fibrous plant material; an aerosol former; and
an active agent absorbed or adsorbed in or on the fibrous plant material, the active agent comprising at least a compound having a structure according to Formula I, Formula II, or Formula III: 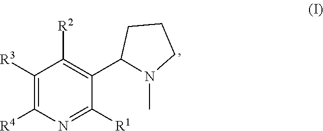
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or
the substituted 3-(1-methylpyrrolidin-2-yl)pyridine has a structure according to Formula II: 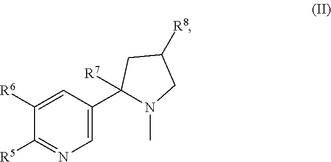
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R⁸ is selected from the group consisting of hydrogen and C₁-C₃ alkyl; and
at least one of R⁷ and R⁸ is not hydrogen; or 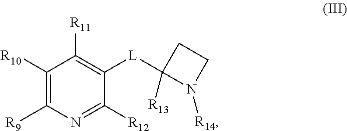
wherein
L is a bond or —OCH₂—*, where the asterisk indicates an attachment point to the azetidine ring;
R⁹, R¹⁰, R¹¹, and R¹² are each independently selected from the group consisting of hydrogen, alkyl, alkoxy, halogen, and cyano;
R¹³ is H or CH₃; and
R¹⁴ is H or CH₃.

2. The aerosol generating material of claim 1, wherein the active agent comprises a compound having a structure according to Formula I, and wherein R¹, R², and R³ are each H.

3. The aerosol generating material of claim 2, wherein R⁴ is optionally substituted C₁-C₆ alkyl, F, Cl, Br, OCH₃, OCH₂CH₃, or CN.

4. The aerosol generating material of claim 3, wherein R⁴ is C₁-C₃ alkyl, such as wherein R⁴ is CH₃.

5. The aerosol generating material of claim 1, wherein the active agent comprises a compound having a structure according to Formula II, and wherein R⁵ and R⁶ are H; R⁷ is CH₃; and R⁸ is H.

6. The aerosol generating material of claim 1, wherein the active agent comprises a compound having a structure according to Formula II, and wherein R⁵, R⁶ and R⁷ are H, and R⁸ is CH₃.

7. The aerosol generating material of claim 1, comprising the fibrous plant material in an amount from about 70% to about 95% by weight, based on a total weight of the aerosol generating material on a dry weight basis.

8. The aerosol generating material of claim 1, wherein the fibrous plant material comprises a tobacco material.

9. The aerosol generating material of claim 1, wherein the fibrous plant material comprises a non-tobacco botanical material.

10. The aerosol generating material of claim 9, wherein the non-tobacco botanical material is a reconstituted non-tobacco botanical material.

11. The aerosol generating material of claim 1, wherein the fibrous plant material comprises a non-tobacco botanical material and the aerosol generating material further comprises a filler.

12. The aerosol generating material of claim 11, wherein the filler comprises a tobacco material, wood fibers, wood pulp, or a combination thereof.

13. The aerosol generating material of claim 1, wherein the non-tobacco botanical material is selected from the group consisting of fennel, star anise, mint, Aspalathus linearis (rooibos), and combinations thereof.

14. The aerosol generating material of claim 1, wherein the aerosol generating material is substantially free of tobacco material.

15. The aerosol generating material of claim 1, wherein the aerosol generating material is substantially free of tea leaves, cassia seeds, and lotus leaves.

16. The aerosol generating material of claim 1, wherein the aerosol generating material is substantially free of 2-methyl-5-(1-methylpyrrolidin-2-yl)pyridine.

17. The aerosol generating material of claim 1, wherein the active agent further comprises a nicotine component, a cannabinoid, a terpene, caffeine, an amino acid, a vitamin, melatonin, a botanical extract, or a combination thereof.

18. The aerosol generating material of claim 1, wherein the active agent further comprises a nicotine component.

19. The aerosol generating material of claim 1, wherein the aerosol generating material is substantially free of a nicotine component.

20. The aerosol generating material of claim 1, further comprising a flavorant.

21. The aerosol generating material of claim 1, further comprising an organic acid.

22. The aerosol generating material of claim 21, wherein the organic acid comprises lactic acid, levulinic acid, benzoic acid, citric acid, 2-methylbutyric acid, 2-methylvaleric acid, or a combination thereof.

23. The aerosol generating material of claim 1, wherein the aerosol former comprises water, polyhydric alcohols, polysorbates, sorbitan esters, fatty acids, fatty acid esters, waxes, cannabinoids, terpenes, sugar alcohols, or a combination thereof.

24. The aerosol generating material of claim 1, comprising the aerosol former in an amount from about 10% to about 20% by weight of the aerosol generating material.

25. The aerosol generating material of claim 1, wherein a ratio by weight of the aerosol former to the fibrous plant material is in a range from about 1:7 to about 1:3.

26. An aerosol generating rod comprising the aerosol generating material of claim 1.

27. An article comprising the aerosol generating material of claim 1 or the aerosol generating rod according to claim 26.

28. The article of claim 27, comprising an aerosol generating section comprising the aerosol generating material or the aerosol generating rod.

29. The article of claim 27, comprising a wrapper circumscribing the aerosol generating material or the aerosol generating rod.

30. A delivery system comprising the aerosol generating material of claim 1, or the aerosol generating rod of claim 26.

31. The delivery system of claim 30, wherein the aerosol generating material is in the form of cut strips.

32. A method of preparing an aerosol generating material comprising a fibrous plant material; an aerosol former; optionally, a binder, a filler, or both a binder and a filler; and an active agent absorbed or adsorbed in or on the fibrous plant material, the active agent comprising at least a substituted 3-(1-methylpyrrolidin-2-yl)pyridine having a structure according to Formula I or Formula II, the method comprising:
providing a sheet of aerosol generating material, the sheet comprising a fibrous plant material; an aerosol former; optionally, a binder, a filler, or both a binder and a filler; and
applying the substituted 3-(1-methylpyrrolidin-2-yl)pyridine to the sheet of aerosol generating material.

33. A method of preparing a plurality of aerosol generating rods, comprising:
a) providing an aerosol generating material comprising a fibrous plant material and an aerosol former, the aerosol generating material being in the form of a sheet, an extrudate, or a shredded material;
b) conveying the aerosol generating material to a rod formation device;
c) applying a substituted 3-(1-methylpyrrolidin-2-yl)pyridine to the aerosol generating material as the aerosol generating material is conveyed to the rod formation device;
d) forming the aerosol generating material into an enwrapped rod having an outer wrapper circumferentially wrapped around the aerosol generating material; and
e) cutting the enwrapped rod into discrete lengths to form the plurality of aerosol generating rods.

34. The method of claim 33, further comprising attaching a filter element to each of the plurality of aerosol generating rods.

35. The method of claim 33, wherein the substituted 3-(1-methylpyrrolidin-2-yl)pyridine has a structure according to Formula I: 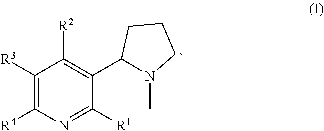
wherein R¹, R², R³, and R⁴ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted; and wherein at least one of R¹, R², R³, and R⁴ are not hydrogen; or
the substituted 3-(1-methylpyrrolidin-2-yl)pyridine has a structure according to Formula II: 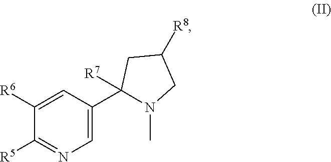
wherein:
R⁵ and R⁶ are each independently selected from the group consisting of hydrogen, alkyl, alkoxy, cycloalkyl, alkenyl, alkynyl, aryl, alkylaryl, amino, halogen, and cyano, wherein any of said alkyl, alkoxy, cycloalkyl, alkenyl, alkenyl, alkynyl, aryl, alkylaryl, and amino may optionally be substituted;
R⁷ is selected from the group consisting of hydrogen and CH₃;
R¹ is selected from the group consisting of hydrogen and C₁-C₃ alkyl; and
at least one of R⁷ and R⁸ is not hydrogen.

36. The method of claim 33, wherein the applying comprises metered dosing of the substituted 3-(1-methylpyrrolidin-2-yl)pyridine in a range from about 0.1 to about 5 mg per aerosol generating rod, such as a range of from about 0.3 to about 2 mg or about 0.5 mg to about 1.5 mg per aerosol generating rod, and optionally wherein the variance in metered dosing for each aerosol generating rod is no more than +/−10%.
